(19) United States
(12) Patent Application Publication
(10) Pub. No.: US 2004/0224995 A1
(43) Pub. Date: Nov. 11, 2004

(54) Neuroprotective effects of PPARy agonists against cellular oxidative insults

(75) Inventors: James W. Simpkins, Fort Worth, TX (US); Paul Aoun, Fort Worth, TX (US)

Correspondence Address: JACKSON WALKER LLP, 2435 NORTH CENTRAL EXPRESSWAY, SUITE 600, RICHARDSON, TX, 75080, US

(73) Assignee: University of North Texas Health Science Center at Fort Worth, Fort Worth, TX

(21) Appl. No.: 10/435,501

(22) Filed: May 9, 2003

(51) Int. Cl. A61K 31/426

(52) U.S. Cl. 514/369; 514/456

(57) ABSTRACT

The current invention comprises compositions and methods for protecting a neuronal cell of a subject from a toxic insult. The method includes delivering an effective amount of a neuroprotective compound to the neuronal cells before or after the toxic insult. The neuroprotective compounds contain a peroxisome proliferator activated receptor (“PPAR-γ”) binding moiety with either a phenolic ring moiety or a prostaglandin (“PG”) with a reactive α,β-unsaturated carbonyl group on the cyclopentenone ring. Other novel compounds are also disclosed. The toxic insult that impinges upon the neuronal cell may be an acute process, or chronic disease process. Oxidative stress (e.g. hydrogen peroxide, and glutamate), injury, and secondary physiological responses to injury are among the acute processes discussed. Clinical disease processes that comprise oxidative stress, inflammatory responses, strokes, Alzheimer's disease, dementia, and Parkinson's disease are also addressed. Because PPAR-γ agonists are used to treat type II diabetes, a condition that leads to neurological complications, a single agent that can target both conditions is of great therapeutic value.

### BACKGROUND

The current invention pertains to a composition and methods for protecting a neuronal cell of a subject from a toxic insult. The toxic insult that impinges upon the neuronal cell may be an acute process, or chronic disease process. The method includes delivering an effective amount of a neuroprotective compound containing a peroxisome proliferator-activated receptor-gamma (“PPAR-γ”) binding moiety with a phenolic ring moiety to the neuronal cell. An insulin-sensitizing thiazolidinedione (“TZD”) class of drug was found to be an excellent neuroprotective compound. In a specific embodiment, the TZD compound called troglitazone was utilized as a neuroprotective compound. Additionally, another neuroprotective compound comprising a prostaglandin (“PG”) with a reactive α,β-unsaturated carbonyl group on the cyclopentenone ring, and a peroxisome proliferator-activated receptor-gamma (“PPAR-γ”) binding moiety, was also effective to protect neuronal cells from toxic insults. Other novel compounds that comprise the PPAR-γ binding moiety with a phenolic ring moiety, or cyclopentenone ring are also disclosed.

Pathological conditions resulting from the accelerated or ongoing death of neurons are prevalent in today's society and include chronic diseases such as Alzheimer's disease and Parkinson's disease, acute diseases such as stroke, brain cell loss that follows myocardial infarction, and acute neuronal injury associated with spinal cord trauma and head trauma. Chronic and acute neurodegenerative diseases and acute neuronal injury as well as associated mortality and morbidity are largely untreatable. The consequences from these conditions include patient disability, which results a significant reduction in quality of life for the patient, and a financial burden to society resulting from the increased cost of patient care. Effective therapeutic approaches directed to the prevention or reduction of neuron death or damage associated with the above conditions are needed. At present, the greatest challenge in the development of therapeutic agents for treating conditions in the brain resulting from neuron loss include obtaining an efficacious drug that is relatively non-toxic, suitable for use in both females and males, and which can readily access the brain across the blood-brain barrier. Parenteral (e.g. subcutaneous) delivery of the neuroprotective compounds is outlined in preferred embodiments. Because PPAR-γ agonists are used to treat type II diabetes, a condition that leads to neurological complications, a single agent that can target both conditions is of great therapeutic value.

Oxidative stress and neurodegeneration. Oxidative stress is believed to be at the core of many age-related neurodegenerative diseases that result in oxidative damage and eventual death of neuronal cells. For example, the oxidative stress mechanism of Alzheimer's Disease (“AD”), proposes that oxidative damage leads to a pathological cascade, which ultimately results in neuronal cell death and dementia. Free radicals are generated in the central nervous system (“CNS”) by ongoing oxygen metabolism and biological events associated with aging, chronic neurological diseases, injury, and inflammation (20). The brain derives its energy almost exclusively from oxidative metabolism of the mitochondrial respiratory chain, and the leakage of high energy electron along the mitochondrial transport chain causes the formation of superoxide anion (.O₂⁻) and hydrogen peroxide (H₂O₂) (18). In addition, several features of the brain makes it more vulnerable to oxidative stress (19): (a) high oxidative metabolism, (b) polyunsaturated fatty acids that are subjected to peroxidation, (c) iron that catalyzes the formation of ROS and (d) a relatively low level of brain-antioxidants. Additionally, many enzymes in the brain including monoamine oxidase (“MAO”), tyrosine hydroxylase, and L-amino oxidase, produce H₂O₂ as a normal byproduct of their activity. Furthermore, increased concentration of nitric oxide synthase (“NOS”) in certain neurons forms nitric oxide (“NO”) that reacts rapidly with (.O₂⁻) to yield the peroxynitrite anion, which can decompose to (.OH). At the molecular level, the production of reactive oxygen species (“ROS”) is also associated with many forms of apoptosis (17). In recent years, considerable data have accrued indicating that the brain of Alzheimer disease (“AD”) patients is under increased oxidative stress, which may play a role in the pathogenesis of neuronal degeneration (21-26) and death in this disorder (81). Thus, oxidative stress has been implicated in a number of neurodegenerative diseases (e.g. AD, Parkinson (27), and stroke (28)), but few pharmaceuticals, if any, successfully address this problem.

Diabetes mellitus and oxidative stress. Oxidative stress has also been implicated in a number of other non-neuronal diseases (e.g. diabetes). For example, over the past decade, there has been substantial interest in oxidative stress and its potential role in diabetogenesis, development of diabetic complications, atherosclerosis and associated cardiovascular disease. For instance, lipid peroxidation is increased in patients with diabetes mellitus (17). Recent experimental findings also suggest that humans with poorly controlled diabetes mellitus had an overproduction of reactive oxygen- or nitrogen species (“ROS”/“RNS”), lowered antioxidant defenses, and altered enzymatic pathways that contributed to endothelial, vascular, and neurovascular dysfunction (18). Consequently, long-term vascular complications still represent the main cause of morbidity and mortality in diabetic patients (19).

Peroxisome Proliferator-Activated Receptors (“PPAR”) and type II diabetes mellitus. Advances in the understanding of the pathophysiology of type 2 diabetes have led to the identification of new approaches to therapy. Progressive deterioration of glycemic control and a high rate of metabolic and vascular complications are major inevitable consequences of type 2 diabetes. Oral anti-diabetic agents are prescribed for their anti-hyperglycemic activity, but often fail to maintain glycemic control for extended periods of time (14). Peroxisome proliferator-activated receptors (“PPAR”) agonists in the thiazolidinedione (“TZD”) class of drug have been shown to improve insulin sensitivity and maintain glycemic control over an extended period of time (15). In addition, insulin resistance is linked with a cluster of co-morbid conditions such as dyslipedemia, abdominal obesity, hypertension, and abnormalities of the fibronolytic system (16). A growing body of evidence is suggesting that PPAR agonists have a positive impact on improving these conditions (14), and may also play a protective role in neurodegenerative disorders.

The peroxisome proliferator-activated receptors (“PPAR”) belong to the nuclear hormone receptor superfamily. Three different subtypes of PPAR (alpha, beta or delta, and gamma) are coded by three separate genes that have been already been identified in rodents and human (Lemberger et al., 1996). PPAR-alpha is highly expressed in the liver, and mediates the induction of enzymes of the peroxisomal fatty-acid oxidation pathways (Dreyer et al., 1992). PPAR-beta or delta is ubiquitously expressed in a broad range of mammalian tissues (Krey et al., 1997) and in the adult rat (Lemberger et al., 1996). PPAR-gamma regulates the process of adipogenesis (Chawla et al., 1994; Tontonoz et al., 1994). Analysis of the promoter of several PPAR target genes revealed a consensus PPAR responsive element (“PPRE”) as a direct repeat of two AGGTCA half-sites separated by a single intervening nucleotide (DR-1) (10). By itself, PPAR exhibits a low affinity for DNA; high affinity binding requires heterodimerization with RXR, the 9-cis-retinoic acid receptor (11). The latter property has allowed the inventors to test whether an increase in the neuroprotective effects of PPAR ligands occurs in the presence of 9-cis-retinoic acid. Although not wanting to be bound by theory, the results of such experiments have allowed the inventors to determine whether neuroprotection by PPAR ligands was PPAR dependent or not. Most of the PPAR target genes that have been identified to date, code for enzymes involved in important pathways of lipid metabolism (12). These metabolic pathways comprise activation of free fatty acids to acyl-CoA derivatives, peroxisomal and mitochondrial β-oxidation, microsomal ω-oxidation, ketogenesis, glyceroneogenesis, fatty acid extracellular transport, cellular uptake and intracellular binding (13).

PPAR-γ is the target for the insulin-sensitizing thiazolidinediones (“TZD's”) (i.e., ciglitazone and troglitazone) class of drugs (Lehmann et al., 1995). TZD's also inhibit the proliferation, hypertrophy, and migration of vascular smooth muscle cells (“VSMC”) induced by numerous growth factors (Dubey et al., 1993; Law et al., 1996). In addition to synthetic ligands, 15-deoxy-Δ^{12,14}-PGJ2 (15d-PGJ2) appears to be a natural ligand for PPARγ with an EC₅₀=7.0 μM (Kliewer et al., 1995). We investigated the role that various PPAR ligands play in protecting neurons against glutamate, H₂O₂, and serum deprivation insults. Herein, the inventors show that both 15d-PGJ2 and troglitazone protect neuronal cells against glutamate and H₂O₂ insults.

PPAR-γ and neuroprotection. Although PPAR-γ agonists are used to treat type II diabetes mellitus, little has been done to explore the role of PPAR-γ agonists in protecting the nervous system against oxidative damage generated during diabetes or under unaltered metabolic status. Studies on the role of PPAR-γ in neuronal survival against other types of insults were also confined. A study by Nishijima et al. has shown that troglitazone improved survival of rat motoneurons against brain-derived neurotrophic factor (“BDNF”) withdrawal but suggested that it did not promote the survival of hippocampal neurons (29). Studies on neuronal survival have explored the role of PPAR-γ in inflammation. Heneka et al. showed that PPAR-γ agonists protected cerebellar granule cells from cytokine-induced apoptotic cell death by inhibition of inducible nitric oxide synthase (“iNOS”) (30). Combs et al. demonstrated that PPAR-γ agonists inhibited the β-amyloid-stimulated secretion of pro-inflammatory products by microglia and monocytes responsible for neurotoxicity and astrocyte activation (31). Thus, antidiabetic drugs with antioxidant properties (e.g. PPAR agonists) have great value in the treatment of diabetes, diabetic complications, and neurodegenerative disorders.

The invention described herein represents the first demonstration of a protective effect of PPAR-γ ligands against oxidative stress in a neuronal cell. The importance of the inventors work lies in this initial discovery, and in showing that 15d-PGJ2 and troglitazone displayed different properties in their neuroprotective effects. Although not wanting to be bound by theory, evidence from these experiments suggest that even though 15d-PGJ2 and troglitazone are both PPAR-γ ligands, their neuroprotective effects may be mediated through a novel pathway(s) independent of the PPAR receptor.

### SUMMARY

One aspect of the current invention is a method for protecting a neuronal cell of a subject from a toxic insult. The method includes delivering an effective amount of a neuroprotective compound containing a peroxisome proliferator-activated receptor-gamma (“PPAR-γ”) binding moiety with a phenolic ring moiety to the neuronal cell. An insulin-sensitizing thiazolidinedione (TZD”) class of drug was found to be an excellent neuroprotective compound. In a specific embodiment, the TZD compound called troglitazone was utilized as a neuroprotective compound. Other compounds that comprise the PPAR-γ binding moiety with a phenolic ring moiety are also disclosed. The toxic insult that impinges upon the neuronal cell may be an acute process, or chronic disease process. Oxidative stress (e.g. hydrogen peroxide, and glutamate), injury, and secondary physiological responses to injury are among the acute processes discussed. Clinical disease and neurodegenerative disorders are addressed. Clinical disease and neurodegenerative disorders defined in specific embodiments include, but are not limited to disorders having progressive loss of neurons that occur either in the peripheral nervous system or in the central nervous system. Examples of neurodegenerative disorders include: chronic neurodegenerative diseases such as Alzheimer's disease, Parkinson's disease, Huntington's chorea, Pick's disease, diabetic peripheral neuropathy, multiple sclerosis, amyotrophic lateral sclerosis; aging; and acute neurodegenerative disorders including: stroke, traumatic brain injury, schizophrenia, peripheral nerve damage, hypoglycemia, spinal cord injury, epilepsy, and anoxia and hypoxia. Parenteral (e.g. subcutaneous) delivery of the neuroprotective compounds is outlined in a preferred embodiment.

Another aspect of the current invention is a method for protecting a neuronal cell of a subject from a toxic insult. The method includes delivering an effective amount of a neuroprotective compound comprising a prostaglandin (“PG”) with a reactive α,β-unsaturated carbonyl group on the cyclopentenone ring, and a peroxisome proliferator-activated receptor-gamma (“PPAR-γ”) binding moiety. In a specific embodiment, the 15-deoxy-Δ^{12,14}-pGJ₂ was utilized as a neuroprotective compound. Other compounds that comprise the PPAR-γ binding moiety with a reactive unsaturated carbonyl group on the cyclopentenone ring are also disclosed. The toxic insult that impinges upon the neuronal cell may be an acute process, or chronic disease process. Oxidative stress (e.g. hydrogen peroxide, and glutamate), injury, and secondary physiological responses to injury are among the acute processes discussed. Clinical disease and neurodegenerative disorders are addressed. Clinical disease and neurodegenerative disorders defined in specific embodiments include, but are not limited to disorders having progressive loss of neurons that occur either in the peripheral nervous system or in the central nervous system. Examples of neurodegenerative disorders include: chronic neurodegenerative diseases such as Alzheimer's disease, Parkinson's disease, Huntington's chorea, Pick's disease, diabetic peripheral neuropathy, multiple sclerosis, amyotrophic lateral sclerosis; aging; and acute neurodegenerative disorders including: stroke, traumatic brain injury, schizophrenia, peripheral nerve damage, hypoglycemia, spinal cord injury, epilepsy, and anoxia and hypoxia. Parenteral (e.g. subcutaneous) delivery of the neuroprotective compounds is outlined in a preferred embodiment.

A third aspect of the current invention are compositions comprising neuroprotective compounds having general structure (III) or (IV):

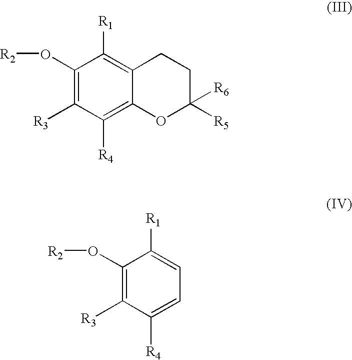

wherein the compositions is used for protecting a neuronal cell of a subject from a toxic insult, and R₁ is a hydrogen or a methyl group; R₂ is hydrogen; R₃ is hydrogen, methyl group, or tertiary butyl group, R₄ is hydrogen or methyl group; R₅ is hydrogen or methyl group; and R₆ is hydrogen, an alkoxy-benzyl group, or an alkoxy benzyl thiazolidinedion group. The composition may further comprise a pharmaceutically acceptable salt, a hydrate, a ester, a solvate, a stereoisomer, or mixtures of stereoisomers of the neuroprotective compound. The toxic insult that impinges upon the neuronal cell may be an acute process, or chronic disease process. Other compounds that comprise the PPAR-γ binding moiety with a phenolic ring moiety are also disclosed. The toxic insult that impinges upon the neuronal cell may be an acute process, or chronic disease process. Oxidative stress (e.g. hydrogen peroxide, and glutamate), injury, and secondary physiological responses to injury are among the acute processes discussed. Clinical disease and neurodegenerative disorders are addressed. Clinical disease and neurodegenerative disorders defined in specific embodiments include, but are not limited to disorders having progressive loss of neurons that occur either in the peripheral nervous system or in the central nervous system. Examples of neurodegenerative disorders include: chronic neurodegenerative diseases such as Alzheimer's disease, Parkinson's disease, Huntington's chorea, Pick's disease, diabetic peripheral neuropathy, multiple sclerosis, amyotrophic lateral sclerosis; aging; and acute neurodegenerative disorders including: stroke, traumatic brain injury, schizophrenia, peripheral nerve damage, hypoglycemia, spinal cord injury, epilepsy, and anoxia and hypoxia. Parenteral (e.g. subcutaneous) delivery of the neuroprotective compounds is outlined in a preferred embodiment.

A fourth aspect of the current invention are compositions comprising neuroprotective compounds having general structure comprising a prostaglandin (“PG”) with a reactive α,β-unsaturated carbonyl group on the cyclopentenone ring, and a peroxisome proliferator-activated receptor-gamma (“PPAR-γ”) binding moiety. In a specific embodiment, the 15-deoxy-Δ^{12,14}-pGJ₂ was utilized as a neuroprotective compound. The composition may further comprise a pharmaceutically acceptable salt, a hydrate, a ester, a solvate, a stereoisomer, or mixtures of stereoisomers of the neuroprotective compound. The toxic insult that impinges upon the neuronal cell may be an acute process, or chronic disease process. Other compounds that comprise the PPAR-γ binding moiety with a phenolic ring moiety are also disclosed. The toxic insult that impinges upon the neuronal cell may be an acute process, or chronic disease process. Oxidative stress (e.g. hydrogen peroxide, and glutamate), injury, and secondary physiological responses to injury are among the acute processes discussed. Clinical disease and neurodegenerative disorders are addressed. Clinical disease and neurodegenerative disorders defined in specific embodiments include, but are not limited to disorders having progressive loss of neurons that occur either in the peripheral nervous system or in the central nervous system. Examples of neurodegenerative disorders include: chronic neurodegenerative diseases such as Alzheimer's disease, Parkinson's disease, Huntington's chorea, Pick's disease, diabetic peripheral neuropathy, multiple sclerosis, amyotrophic lateral sclerosis; aging; and acute neurodegenerative disorders including: stroke, traumatic brain injury, schizophrenia, peripheral nerve damage, hypoglycemia, spinal cord injury, epilepsy, and anoxia and hypoxia. Parenteral (e.g. subcutaneous) delivery of the neuroprotective compounds is outlined in a preferred embodiment.

### BRIEF DESCRIPTION OF THE DRAWINGS

FIG. 1 shows the effects of WY14,643, a PPARα ligand, on HT-22 viability during glutamate exposure;

FIG. 2 shows the effects of PPARβ agonists on HT-22 cell viability during glutamate exposure;

FIG. 3 shows the effects of PPARγ agonists on HT-22 cell viability during glutamate exposure;

FIG. 4 shows the effects of PPARγ agonists on HT-22 cell viability during hydrogen peroxide (H₂O₂) exposure;

FIG. 5 shows the effects of preincubation time on the neuroprotection by PPARγ agonists in HT-22 cells during glutamate exposure;

FIG. 6 shows the effects of post-treatment with troglitazone on HT-22 cell viability during glutamate exposure;

FIG. 7 shows the effects of pre-incubation time on the neuroprotection by PPARγ agonists on HT-22 cell viability during H₂O₂ exposure;

FIG. 8 shows cytoplasmic extracts from HT-22 and SK—N—SH that were subjected to Western blot analysis using an antibody to the PPARγ receptor (48 kD);

FIG. 9 shows the effects of 15d-PGJ2,9-cis-retinoic acid, and their combination on HT-22 cell viability during glutamate exposure;

FIG. 10 shows the neuroprotective effects of PPAR-γ ligands against glutamate cytotoxicity in HT-22 cells;

FIG. 11 shows the neuroprotective effects of PPAR-γ ligands against glutamate cytotoxicity in C6 Glioma cells;

FIG. 12 shows the neuroprotective effects of PPAR-γ ligands against glutamate cytotoxicity in RGC-5 cells;

FIG. 13 shows the effects of PPAR-γ agonists on HT-22 cell viability during H₂O₂ (30 μM) exposure;

FIG. 14 shows the effects of troglitazone on HT-22 and RGC-5 cell viability during BSO exposure;

FIG. 15 shows the effects of troglitazone on HT-22 and RGC-5 cell viability during BSO exposure;

FIG. 16 shows the effects of 15dPGJ2 and troglitazone medium withdrawal on neuroprotection in HT-22 cells during glutamate (15 mM) exposure;

FIG. 17 shows several structures of cyclopentenone prostaglanin's;

FIG. 18 shows an illustration of a cell with a signaling cascade that expresses NFκB responsive genes;

FIG. 19 shows a vitamin E moiety and several thiazolidinedione compounds;

FIG. 20 shows known, and novel TZD's (“nTZD's”) compounds;

FIG. 21 shows the neuroprotective effects of 15d-PGJ2 on HT-22 cell viability during glutamate exposure;

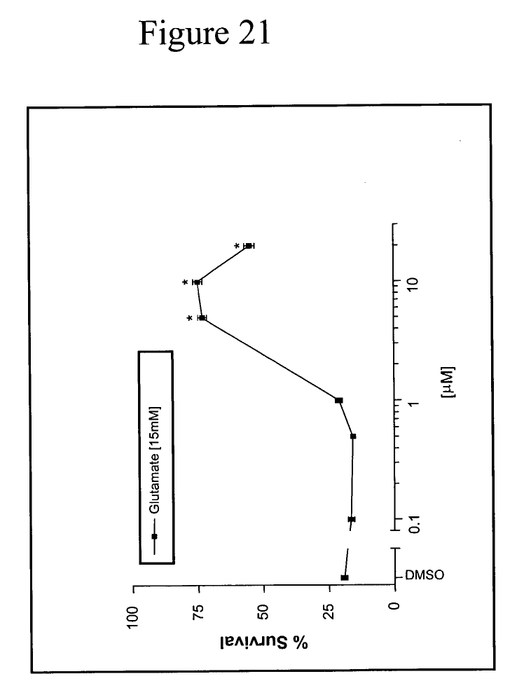

FIG. 22 shows micrographs of control cells, glutamate treated cells (15 mM), and glutamate+15dPGJ2 (5 uM) treated cells;

FIG. 23 shows the neuroprotective effects of 15d-PGJ2 on HT-22 cell viability during H₂O₂ exposure;

FIG. 24 shows the effects of preincubation time on the neuroprotection by 15d-PGJ2 on HT-22 cell viability;

FIG. 25 shows the neuroprotective effects of 15d-PGJ2 on HT-22 cell viability during glutamate exposure are likely independent of PPARγ receptor;

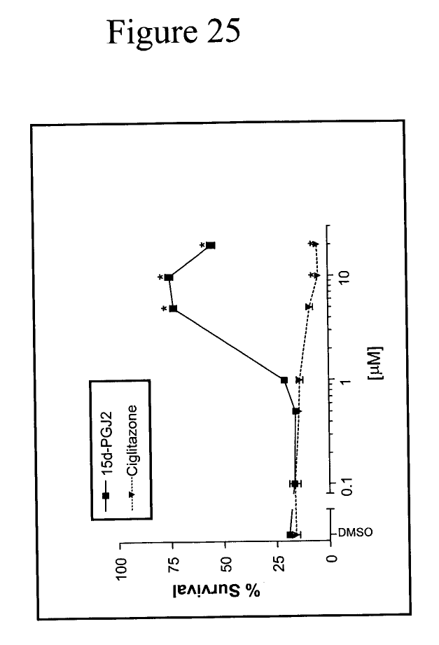

FIG. 26 shows several structures of cyclopentenone prostaglandins;

FIG. 27 shows the neuroprotective effects of 15d-PGJ2, PGA2 in HT-22 cells during glutamate exposure are dependent on the reactive α, β unsaturated group of the cyclopentenone ring;

FIG. 28 shows 15d-PGJ2 upregulates the catalytic (C) and regulatory (R) subunits of γ-glutamyl-cysteine synthetase (“GCS”), the rate limiting enzyme in glutathione synthesis;

FIG. 29 shows 15d-PGJ2 upregulates the catalytic (C) and regulatory (R) subunits of γ-glutamyl-cysteine synthetase (“GCS”) the rate limiting enzyme in glutathione synthesis, independent of glutamate; and

FIG. 30 shows that a 12 h and a 2 h subcutaneous (s.c.) injection of troglitazone to ovariectomized female Sprague Dawley rats before the onset of middle cerebral artery occlusion (“MCAO”) reduced the ischemic lesion area by about 50% compared to control animals.

### DESCRIPTION OF PREFERRED EMBODIMENTS

Terms:

The term “delivery” as used herein is defined as a means of introducing a material into a subject, a cell or any recipient, by means of chemical or biological process, injection, mixing, electroporation, sonoporation, or combination thereof, either under or without pressure.

The term “functional biological equivalent” of a neuroprotective compound as used herein is a compound that has been engineered to contain a distinct chemical structure to the peroxisome proliferator-activated receptor-gamma (“PPAR-γ”) binding moiety with a phenolic ring moiety, or prostaglandin (“PG”) with a reactive α,β-unsaturated carbonyl group on the cyclopentenone ring moiety, and a peroxisome proliferator-activated receptor-gamma (“PPAR-γ”) binding moiety, but simultaneously having similar or improved biologically activity when compared to these claimed structures.

The term “peroxisome proliferator activated receptor gamma (“PPAR-γ”) binding moiety” as used herein, refers to a compound the comprises a structural component that binds to a PPAR-γ.

The term “subject” as used herein refers to any species of the animal kingdom. In preferred embodiments it refers more specifically to humans, domesticated animals, and research animals.

The current invention comprises compositions and methods for protecting a neuronal cell of a subject from a toxic insult. The method includes delivering an effective amount of a neuroprotective compound containing a peroxisome proliferator-activated receptor-gamma (“PPAR-γ”) binding moiety with a phenolic ring moiety to the neuronal cell. An insulin-sensitizing thiazolidinedione (“TZD”) class of drug was found to be an excellent neuroprotective compound. In a specific embodiment, the TZD compound called troglitazone was utilized as a neuroprotective compound. Additionally, another neuroprotective compound comprising a prostaglandin (“PG”) with a reactive α,β-unsaturated carbonyl group on the cyclopentenone ring, and a peroxisome proliferator-activated receptor-gamma (“PPAR-γ”) binding moiety, was also effective to protect neuronal cells from toxic insults. Other novel compounds that comprise the PPAR-γ binding moiety with a phenolic ring moiety, or cyclopentenone ring are also disclosed. The toxic insult that impinges upon the neuronal cell may be an acute process, or chronic disease process. Oxidative stress (e.g. hydrogen peroxide, and glutamate), injury, and secondary physiological responses to injury are among the acute processes discussed. Clinical disease and neurodegenerative disorders are addressed. Clinical disease and neurodegenerative disorders defined in specific embodiments include, but are not limited to disorders having progressive loss of neurons that occur either in the peripheral nervous system or in the central nervous system. Examples of neurodegenerative disorders include: chronic neurodegenerative diseases such as Alzheimer's disease, Parkinson's disease, Huntington's chorea, Pick's disease, diabetic peripheral neuropathy, multiple sclerosis, amyotrophic lateral sclerosis; aging; and acute neurodegenerative disorders including: stroke, traumatic brain injury, schizophrenia, peripheral nerve damage, hypoglycemia, spinal cord injury, epilepsy, and anoxia and hypoxia.

Pathological conditions resulting from the accelerated or ongoing death of neurons are prevalent in today's society and include chronic diseases such as Alzheimer's disease and Parkinson's disease, acute diseases such as stroke, brain cell loss that follows myocardial infarction, and acute neuronal injury associated with spinal cord trauma and head trauma. Chronic and acute neurodegenerative diseases and acute neuronal injury as well as associated mortality and morbidity are largely untreatable. The consequences from these conditions include patient disability, which results a significant reduction in quality of life for the patient, and a financial burden to society resulting from the increased cost of patient care. Effective therapeutic approaches directed to the prevention or reduction of neuron death or damage associated with the above conditions are needed. At present, the greatest challenge in the development of therapeutic agents for treating conditions in the brain resulting from neuron loss include obtaining an efficacious drug that is relatively non-toxic, suitable for use in both females and males, and which can readily access the brain across the blood-brain barrier. Parenteral (e.g. subcutaneous) delivery of the neuroprotective compounds is outlined in preferred embodiments. Because PPAR-γ agonists are used to treat type II diabetes, a condition which leads to neurological complications, a single agent that can target both conditions is of great therapeutic value.

The present invention demonstrates for the first time that troglitazone (I) and 15-deoxy-Δ^{12,14}-PGJ2 (“15d-PGJ2”) (II) protect mouse hippocampal HT-22 cells against glutamate and H₂O₂ toxicities.

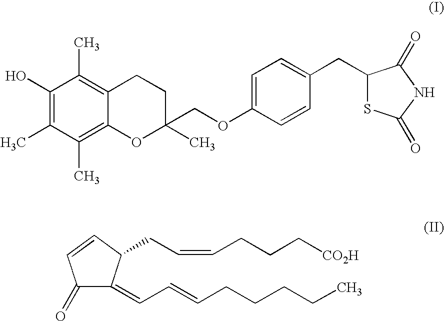

The neuroprotection was dose dependent, effective against two pro-oxidant insults, glutamate and H₂O₂, and selective for 15d-PGJ2 and troglitazone. In contrast, none of the other Peroxisome proliferator-activated receptors (“PPARs”) agonist that were tested (e.g. ciglitazone, PPARα or β/δ agonists) showed neuroprotection. These findings reveal a novel neuroprotective property of PPARγ ligands in neuronal tissues. Additionally, the potent neuroprotective cyclopentenone PG's, TZD's and nTZD's that are protective in neuronal cell lines are also neural protective in animals.

Oxidative stress is believed to be at the core of many age-related diseases that result in oxidative damage and eventual death cells. Recent experimental findings suggest that humans with poorly controlled diabetes mellitus had an overproduction of reactive oxygen- or nitrogen species (“ROS”/“RNS”), lowered antioxidant defenses, and altered enzymatic pathways that contributed to endothelial, vascular, and neurovascular dysfunction (18). Consequently, long-term vascular complications still represent the main cause of morbidity and mortality in diabetic patients (19). Additionally, overproduction of ROS/RNS, or decreased antioxidant capabilities have also been suggested to underline the complications associated with neurodegenerative disorders (e.g. Alzheimer's disease, Parkinson's disease, Huntington's chorea, Pick's disease, diabetic peripheral neuropathy, multiple sclerosis, amyotrophic lateral sclerosis; aging; and acute neurodegenerative disorders including: stroke, traumatic brain injury, schizophrenia, peripheral nerve damage, hypoglycemia, spinal cord injury, epilepsy, and anoxia and hypoxia).

Because peroxisome proliferator-activated receptors (“PPARs”) are involved in regulating many metabolic and inflammatory processes, the present invention demonstrates the role that some PPAR ligands play in protecting neuronal cells from toxic insults. For that purpose, the inventors used WY 14,643 as a PPARα agonist, L-165041 and L-783483 as PPARβ ligands, and troglitazone (I), 15d-PGJ2 (II), and ciglitazone for PPARγ. Some experiments were performed using HT-22, an immortalized mouse hippocampal cell line; SK—N—SH, a human neuroblastoma cell line; and in live rats. Cell viability against glutamate, hydrogen peroxide (H₂O₂), and serum deprivation insults was determined using a calcein AM assay. Of the compounds tested, only 15d-PGJ2 (II) and troglitazone (I) showed a dose-dependent neuroprotection from glutamate and H₂O₂ insults in HT-22 cells. None of the PPAR agonists were protective in SK—N—SH cells. A minimum of four to six hours preincubation with 15d-PGJ2 (II) was required to achieve significant neuroprotrection. On the other hand, troglitazone (I) was protective even when administered simultaneously with glutamate, or for up to eight hours post glutamate insult. To investigate whether the neuroprtective effects are mediated through PPARγ we first determined through western blotting that HT-22 and SK—N—SH cells express PPARγ. Additionally, ovariectomized female rats that were treated with troglitazone and subjected to a middle cerebral artey occlusion (“MCAO”) showed a reduced ischemic neruonal lesion area when compared to non-treated controls.

Although not wanting to be bound by theory, the neuroprotective effects of the compounds tested are unlikely to be mediated through the PPARγ for at least two reasons: 1) Various amounts of other PPARγ agonists (i.e., ciglitazone, L-783483) were not neuroprotective. 2) By itself, PPAR exhibits a low affinity for DNA, and high affinity binding requires heterodimerization with RXR, the 9-cis retinoic acid receptor; administrating 9-cis retinoic acid in conjunction with 15d-PGJ2 did not alter the neuroprotective effects of the latter. Our results further demonstrate a novel neuroprotective effect of 15d-PGJ2 (II) and troglitazone (I) is likely independent of the PPARγ.

Glutathione also plays a role in protecting cells against free radicals, reactive oxygen species, and other toxic compounds generated during oxidative stress. Although not wanting to be bound by theory, the invention uses cell culture models where glutamate, H₂O₂, and BSO induce cell death via the oxidative pathway, and 15d-PGJ2 protected HT-22, C6 glioma, and RGC-5 from these oxidative insults. Therefore, exploring the role of 15d-PGJ2 in maintaining adequate glutathione levels may be valuable in deciphering the mechanism of action of this compound.

The invention may be better understood with reference to the following examples, which are representative of some of the embodiments of the invention, and are not intended to limit the invention.

### EXAMPLE 1

Our initial studies show an in vitro assessment of the neuroprotective effects of PPAR-γ ligands. The in vitro model used for oxidative cell death was based upon glutamate, hydrogen peroxide, and buthionine sulfoximine (“BSO”) administration to HT-22 mouse hippocampal, C6 rat glioma, and RGC-5 retinal ganglion cell lines. The ease that the above cell lines are cultured is well known in the art of cell culture. Additionally, these cell lines are suitable for screening a various number of compounds described in the present invention.

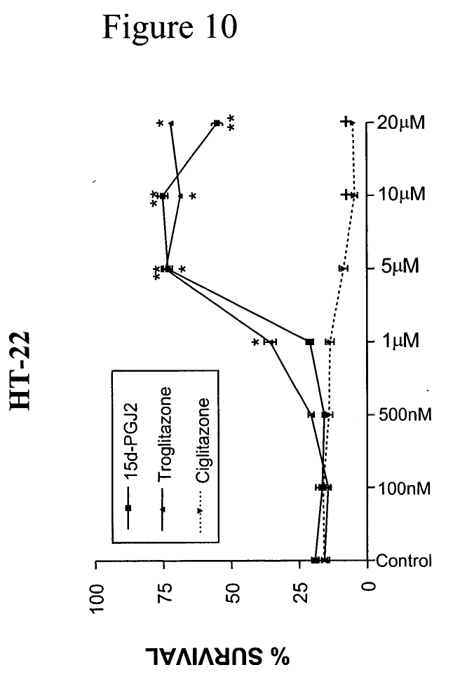

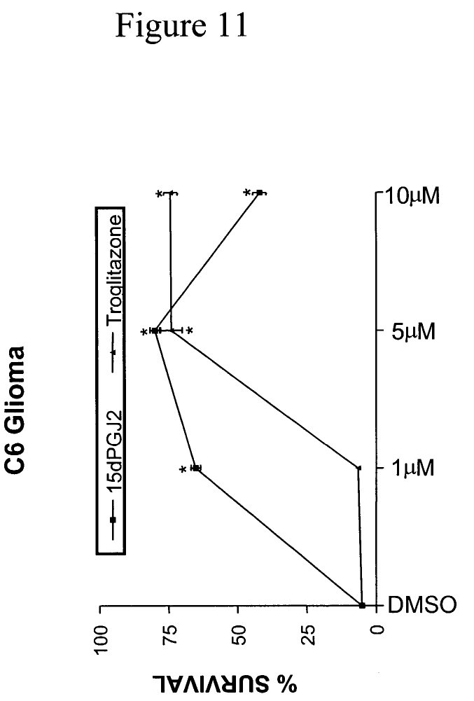

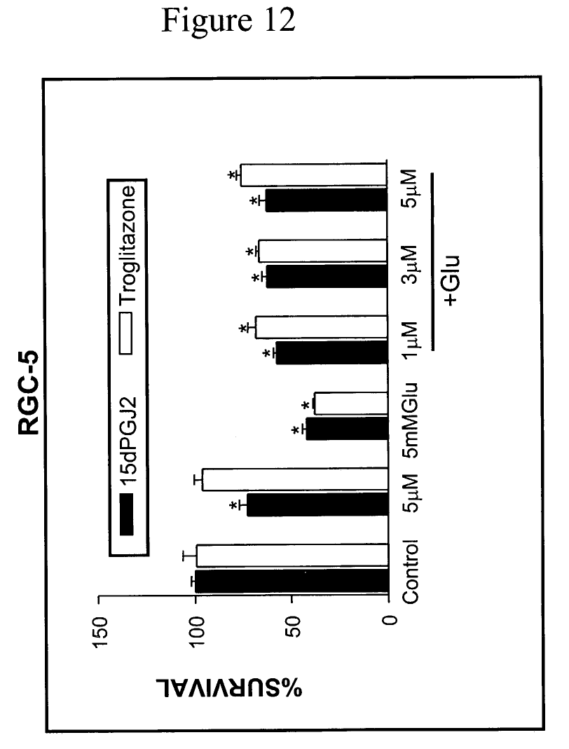

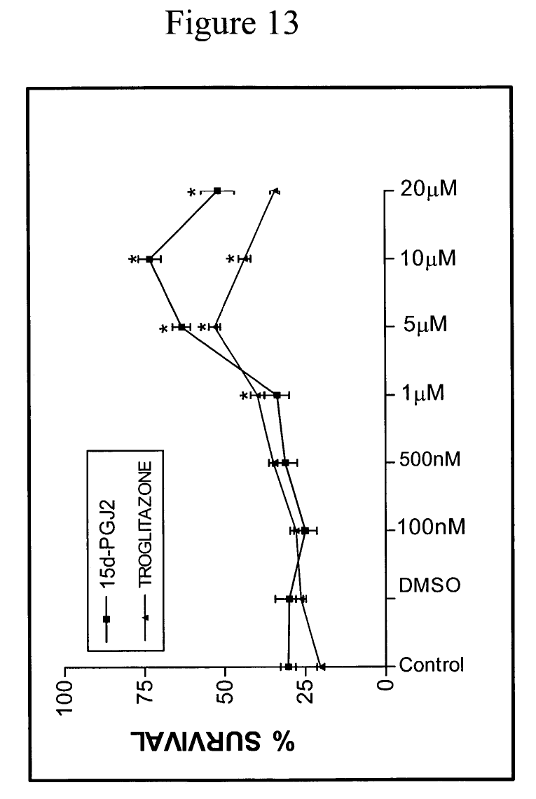

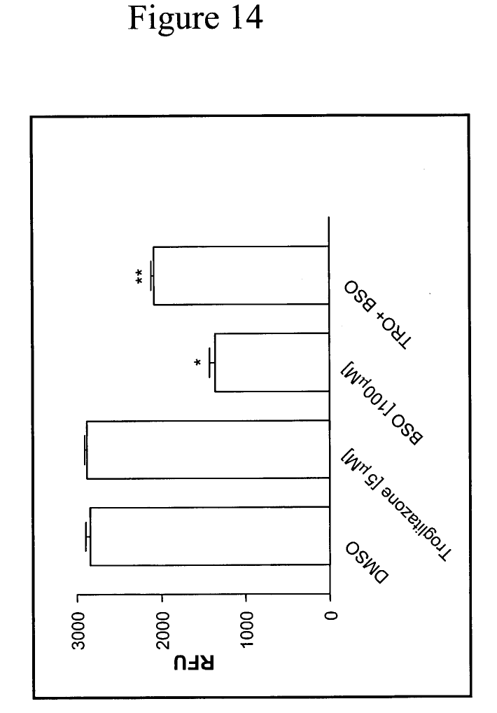

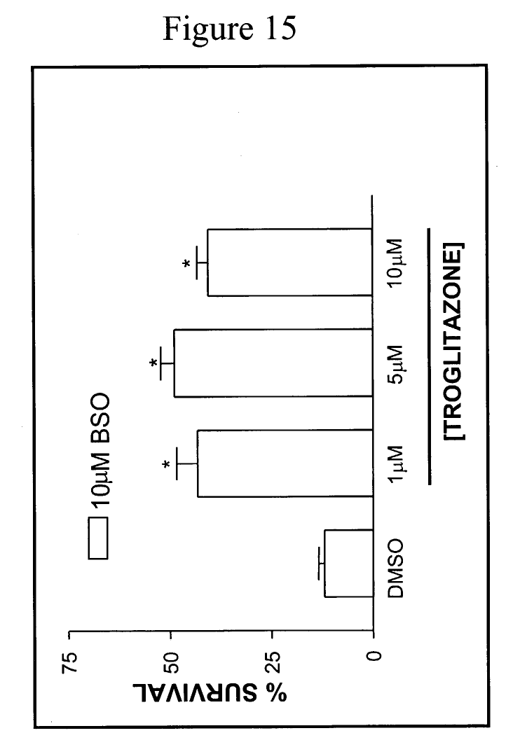

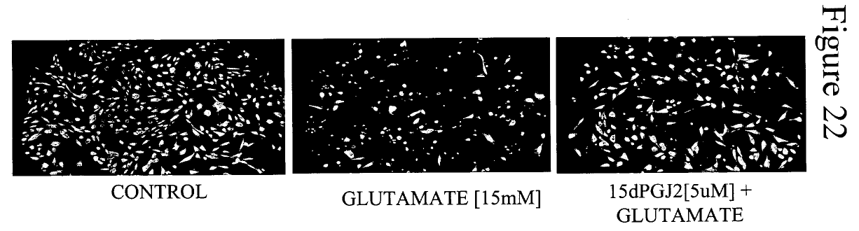

Although not wanting to be bound by theory, the toxicity of glutamate in HT-22 (32) and C6 glioma (33) cell lines is mediated through oxidative stress. HT-22 cells lack ionotropic N-methyl-D-aspartate (“NMDA”) glutamate receptors, and the glutamate-induced cell death appears to occur via a slow onset oxidative stress (32,34,35). Glutamate blocks cystine uptake by inhibiting the glutamate/cystine antiporter in both HT-22 (32) and C6 glioma (33) cell lines. Since cysteine is required for glutathione (“GSH”) synthesis, the intracellular concentration of GSH decreases as a consequence. BSO depletes intracellular glutathione by inhibiting γ-glutamylcysteine synthetase, the rate-limiting enzyme in glutathione synthesis (33). Experimentation has demonstrated that two PPAR-γ agonists, 15d-PGJ2 and troglitazone, protected HT-22 (FIG. 10), C6 glioma, (FIG. 11), and RGC-5 (FIG. 12) cells in a dose dependent manner against glutamate toxicity. The compounds 15d-PGJ2 and troglitazone also protected HT-22 against hydrogen peroxide (FIG. 13). Troglitazone further protected HT-22 (FIG. 14) and RGC-5 (FIG. 15) against BSO. The cell viability was taken at time points around about 14 to 24 hours post-insult time. In some of the experiments, the viability of cells was determined using 2.5 μM calcein AM assay in phosphate-buffered saline (PBS 1×). After 25 minutes of incubation, live cells were distinguished by the presence of intracellular esterase activity, which cleaves the calcein AM dye, producing a bright green fluorescence, as shown in FIG. 22. Viability was measured in Relative Fluorescent Units (“RFU”), and expressed as percentage of vehicle-treated control values.

### EXAMPLE 2

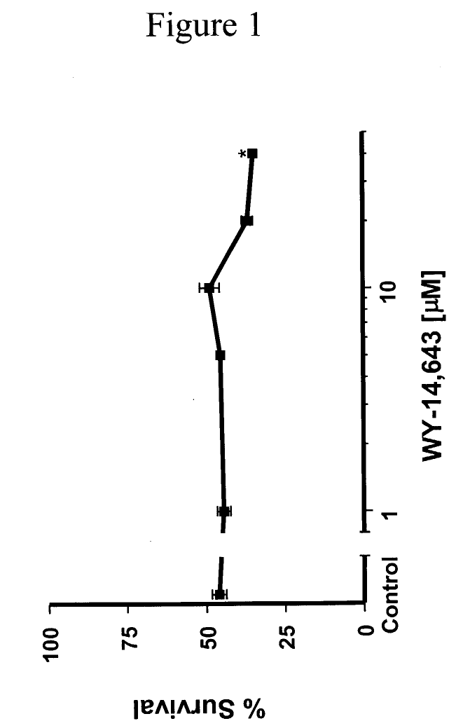

Agonists for the PPARα and PPARβ did not show similar neuroprotective effects compared to those of the two PPARγ ligands. At high concentrations, the PPARα agonist WY-14,643 was toxic. FIG. 1 shows the effects of WY14,643, a PPARα ligand, on HT-22 viability during glutamate exposure. HT-22 cells were preincubated with WY-14643, 24 hrs prior to glutamate insult (20 mM). Cell viability was determined about 14 hours later, and expressed as % survival of the control (non-glutamate-treated cultures). Shown are the mean±SEM for n>6. When error bars are not shown a FIGURE, they are smaller than the symbol used to depict the mean. The asterisk (“*”) indicates p<0.05 vs. respective control. WY14,643 is a potent activator of PPAR alpha (Issemann and Green, 1990; Dreyer et al., 1992) with an approximate EC₅₀ of 1.5 μM (Issemann and Green, 1990). Pretreatment with WY 14,643 at concentrations ranging from 1 to 40 μM failed to prevent glutamate-induced cell death, as shown in FIG. 1. At the highest concentration, WY 14,643 enhanced glutamate toxicity.

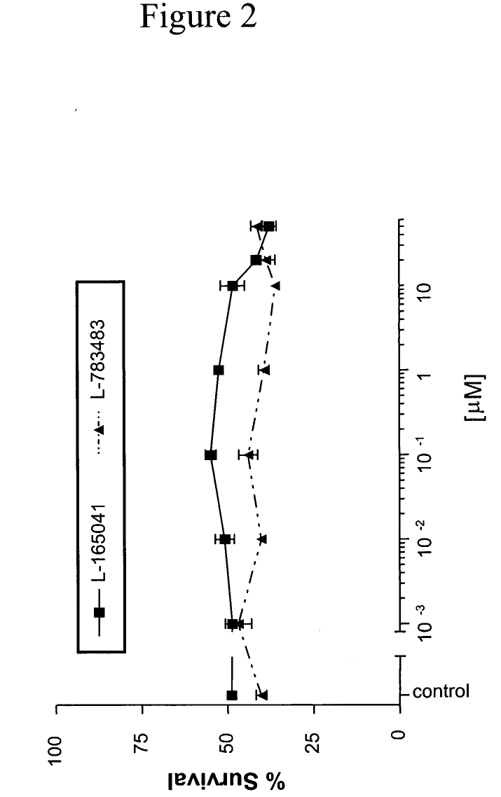

PPARβ (δ) is ubiquitously expressed and particularly abundant in the entire nervous system (Lemberger et al., 1996). The effects of pretreatment with PPARμ (δ) agonists on glutamate toxicity in HT-22 cells can be further demonstrated using L-783483 and L-165041. L-783483 and L-165041 activate PPAR-β with EC₅₀s of approximately 5 nM and 0.5 μM, respectively (Berger et al., 1999). Concentrations of either agonist, ranging from 1 nM to 50 μM, failed to affect glutamate toxicity (FIG. 2). Berger et al. (1999) have shown that at higher concentrations (>51 μM), the PPARβ selective compound, L-165041, was able to induce weak activity of PPARγ. Moreover, L-783483 strongly activated PPARγ (EC₅₀=10 nM) at concentrations close to the EC₅₀ of 5 nM for the activation of PPARβ (5). We tested both L-165041 and L-783483 for neuroprotection at concentrations up to of 50 μM. FIG. 2 shows the effects of PPARβ agonists on HT-22 cell viability during glutamate exposure. HT-22 cells were pretreated with L-165041 or L-783483, 24 hrs prior to glutamate insult (20 mM). To determine the neuroprotective role of other PPAR-γ agonists, we tested another thiazolidinedione, ciglitazone, and a newly identified PPAR-γ ligand, L-783483 against a glutamate insult in HT-22 cells. The affinity of ciglitazone for PPAR-γ is in the low micromolar (μM) range, and similar to that of 15d-PGJ2 (6). L-783483 activates PPAR-γ with an EC₅₀ of 10 nM (5). Pretreatment of with ciglitazone and L-783483 at various concentrations failed to protect HT-22 cells against glutamate toxicity. Although not wanting to be bound by theory, the PPAR agonist's (i.e. WY-14,643, L-165041 and L-783483) inability to protect against glutamate insult provides additional evidence that the neuroprotective effects of 15d-PGJ2 and troglitazone are likely not mediated through PPARγ.

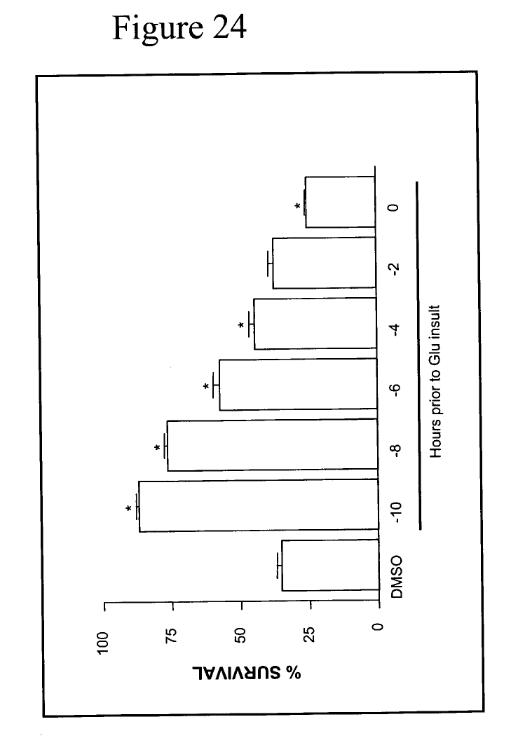

15d-PGJ2 and two TZD's (e.g. troglitazone and ciglitazone) were tested for their ability to protect HT-22 cells against glutamate toxicity. FIG. 24 shows the neuroprotective effects of 15d-PGJ2 on HT-22 viability during glutamate exposure. HT-22 cells were treated with 15-dPGJ2 twenty-four hours prior to a glutamate insult (15 mM). Cell viability was determined about 16 hours later. The data of FIG. 24 was quantified using the calcein AM assay (see FIG. 22). Results are expressed in % survival of the control (non-glutamate-treated) cultures. DMSO received the vehicle only for 15d-PGJ2. Shown are mean±SEM for 6. The asterisk (“*”) indicates p<0.05 vs. respective control. Shown are the neuroprotective effects of 15dPGJ2 against glutamate cytotoxicity.

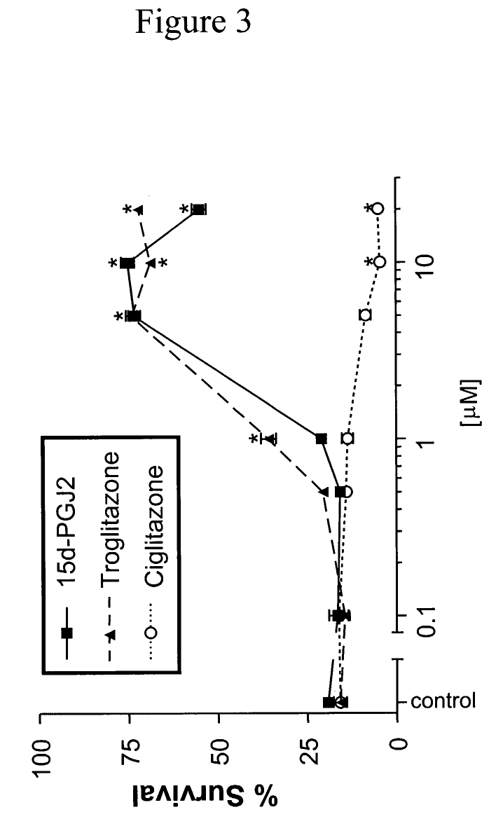

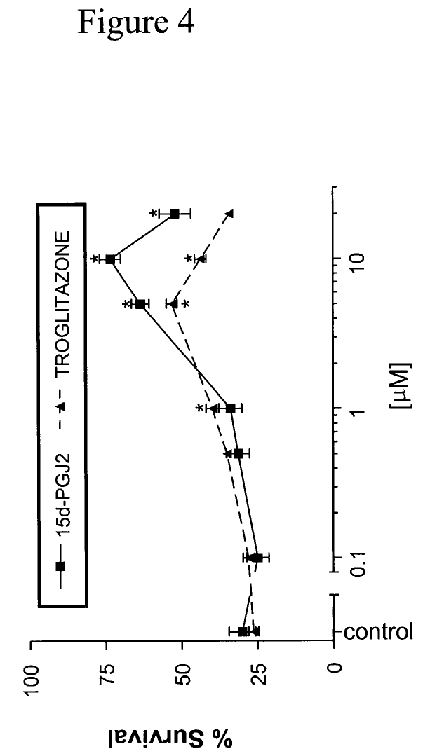

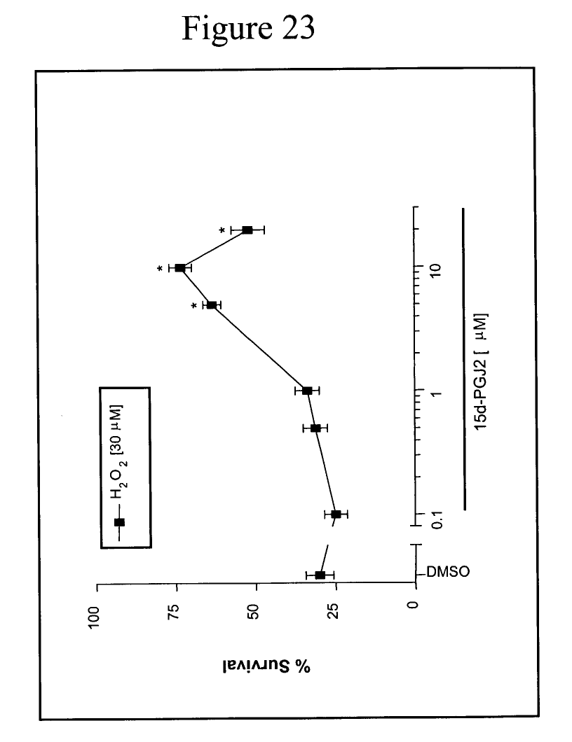

HT-22 cultures were incubated with different concentrations of the three compounds for 24 hours prior to the insult. Two of these compounds, 15d-PGJ2 and troglitazone, showed a dose-dependent protection against glutamate insult (FIG. 3). Peak neuroprotective concentrations for 15d-PGJ2 ranged from 1 to 1 μM. Troglitazone exhibited a dose-dependent neuroprotection over concentrations ranging from 1 μM to 20 μM. For 15d-PGJ2 and troglitazone, toxicity was observed at concentrations at or above 10 μM and 20 μM, respectively. Additionally, we determined the effects of 15d-PGJ2 and troglitazone on HT-22 cells protection against another type of pro-oxidant insult, hydrogen peroxide H₂O₂. As shown in FIG. 4, both compounds showed a dose-dependent protection of HT-22 cells against H₂O₂. Higher concentrations (>10 μM and >20 μM) of 15 dPGJ2 and troglitazone, respectively, were toxic to HT-22 cells. As shown in FIG. 23, the neuroprotective effects of 15d-PGJ2 on HT-22 cell viability occurs during hydrogen peroxide exposure. HT-22 cells were pre-treated with 15d-PGJ2 for 24 hours. Thirty micromolar of H₂O₂ was then administered, and cell viability was determined 24 hours later. The cell viability is expressed as a % survival of the control (non-peroxide treated) cultures. DMSO received the vehicle only for 15d-PGJ2. Shown in FIG. 23 are the mean±SEM for n>6 cultures/group. The asterisk (“*”) indicates p<0.05 versus respective control.

### EXAMPLE 3

The effects of pretreatment of HT-22 cells with PPARγ agonists on serum deprivation toxicity were also studied. We examined whether 15d-PGJ2 or troglitazone have neuroprotective activity against serum deprivation toxicity. HT-22 cells were incubated for 24 hours in 10% FBS medium containing either 15d-PGJ2 or troglitazone. Then, cells were exposed for about 12 hours to a serum free medium with both of the compounds present. 15d-PGJ2 as well as troglitazone failed to protect HT-22 cells against serum deprivation. In addition, 15d-PGJ2 and troglitazone enhanced the serum deprivation-induced toxicity at or above 10 μM and 20 μM, respectively.

Additionally, the effects of pretreatment with PPARγ agonists on H₂O₂ and serum deprivation toxicities were studied in SK—N—SH cells. After showing that protection in HT-22 cells was insult-type specific, we investigated whether it was also cell-type specific. We used SK—N—SH, a human neuroblastoma cell lines, and tested whether 15d-PGJ2 and troglitazone would protect SK—N—SH against H₂O₂ and serum deprivation insults. Neither of the compounds protected SK—N—SH against either insults (data not shown). Higher concentrations (>10 μM and >20 μM) of 15dPGJ2 and troglitazone, respectively, were toxic to SK—N—SH cells.

### EXAMPLE 4

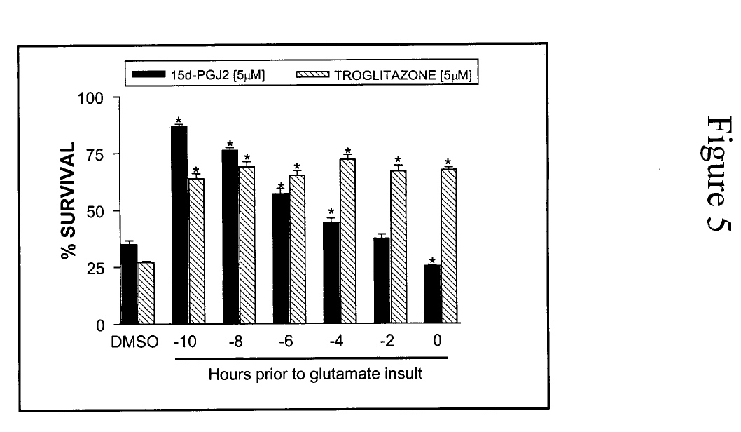

The inventors determined that a minimum pretreatment time with PPARγ agonists may be required to achieve neuroprotection. Our results indicated that 15d-PGJ2 and troglitazone displayed different preincubation time requirement for their neuroprotective effects. For example, HT-22 cells require exposure to 15d-PGJ2 for four to six hours to achieve significant neuroprotection. The effects of preincubation time on the neuroprotection by 15d-PGJ2 on HT-22 cell viability are shown in FIG. 24. HT-22 cells were exposed to 15d-PGJ2 (5 μM) at various times prior to glutamate insult (15 mM). About 15 hours post-insult, cell viability was determined and expressed as % survival of control (non-glutamate-treated cultures). DMSO received vehicle only for 15d-PGJ2. Shown are mean±SEM of n>4 cultures/group. The asterisk (“*”) indicates p<0.05 vs. respective control group. In contrast to 15d-PGJ2, troglitazone was equally protective when administered 0 to 10 hours prior to a glutamate insult (FIG. 5) or H₂O₂ insults.

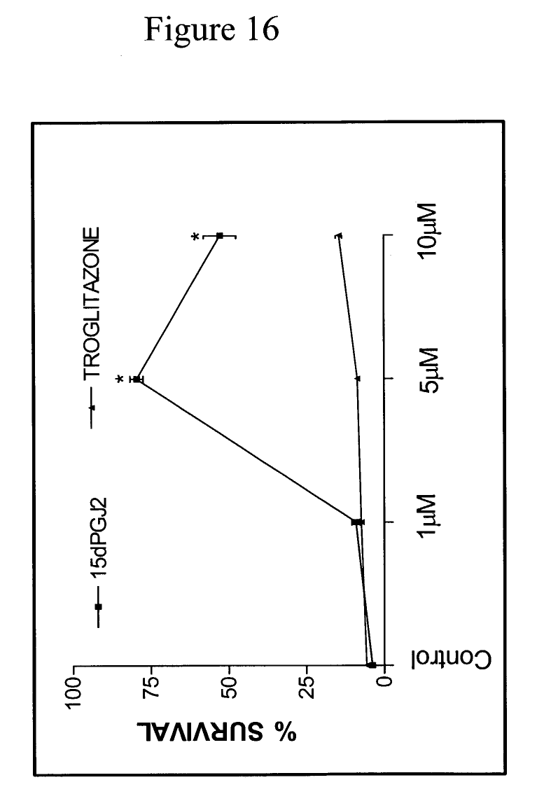

Effects of medium withdrawal on neuroprotection was also monitored. Because of the variability in the preincubation time requirements, the inventors attempted to explore whether the neuroprotective effects of 15dPGJ2 and troglitazone involved the transcriptional and translational machinery in the cells. For that reason, we first administered the compounds in the presence of actinomycin D and cycloheximide, the transcription and translation inhibitors, respectively. Although not wanting to be bound by theory, data from these experiments were inconclusive for two reasons: one, actinomycin D and cycloheximide inhibited cell division and thus cell growth; second, glutamate kill requires RNA and protein synthesis (37). The indirect alternative to that experiment was to preincubate cells with 15d-PGJ2 and troglitazone, and then wash away the medium prior to giving the glutamate insult. Although not wanting to be bound by theory, the rationale is that if either of the PPAR-γ agonists activates the transcriptional and translational cascade of a neuroprotective protein, or inhibits that of a toxic one, then, a sufficient preincubation time with 15d-PGJ2 or troglitazone should provide enough time for that event to occur. Thus, upon the withdrawal of the medium containing the compound, the neuroprotective cascade stimulated by that compound as a result of preincubation is less likely to be altered because the compound has already mediated its effects. In FIG. 16, HT-22 cells were preincubated with 15d-PGJ2 and troglitazone for 24 hours. Then, the medium containing either of the compounds was washed away, and the new media containing the glutamate insult were added. Cells pre-incubated with 15d-PGJ2 were protected in a dose dependent manner after the withdrawal of the compound-contained-medium. There was a loss of neuroprotection when troglitazone was withdrawn.

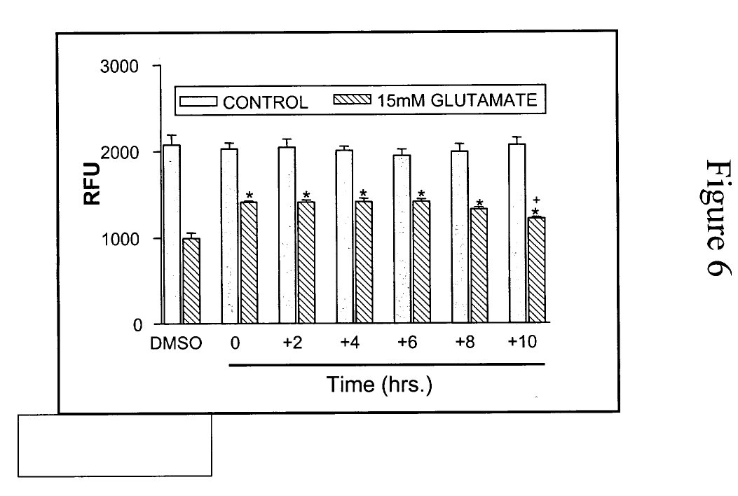

The effects of post-treatment-time on 15d-PGJ2 and troglitazone protection of HT-22 cells from glutamate toxicity was also determined. If added at the same time as glutamate or up to ten hours thereafter, 15dPGJ2 failed to display any neuroprotective effects. Since troglitazone protected HT-22 cells when administered simultaneously with the glutamate insult, the inventors also determined the length of the delay between glutamate exposure and troglitazone treatment that would still afford neuroprotection. For these experiments, glutamate was administered to HT-22 cells, and troglitazone was then added 0 to 10 hours thereafter. The neuroprotective effects of troglitazone remained unaltered up to 6 hours post glutamate insult, as shown in FIG. 6. At 8 and 10 hours post glutamate insult, troglitazone still significantly protected HT-22 cells, but the extent of protection decreased. Although not wanting to be bound by theory, the reason for different preincubation requirements by troglitazone during the glutamate versus the H₂O₂ insults could be explained by the mechanism of cytotoxicity of the two insults. During glutamate exposure, reactive oxygen species (“ROS”) production proceeds in two phases: an initial slow increase for the first 6h, followed by a much higher rate (Tan et al., 1998). H₂O₂ on the other hand rapidly penetrates into cells and induces oxidation of a variety of molecules. In this situation, pretreatment for a time sufficient to allow distribution of the compound and activation of a yet to be determined protective mechanisms is needed.

### EXAMPLE 5

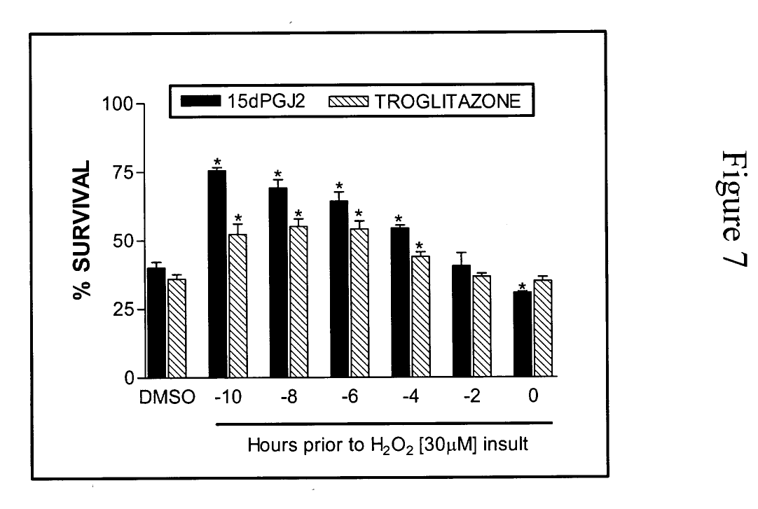

Effects of preincubation-time on PPARγ agonists protection of HT-22 cells from H₂O₂ toxicity were determined. FIG. 7 shows that 4 hours of pre-incubation with either 15dPGJ2 or troglitazone was required to achieve significant neuroprotection from H₂O₂ toxicity in HT-22 cells. In contrast, the effects of post-treatment-time on 15d-PGJ2 and troglitazone protection of HT-22 cells from H₂O₂ toxicity were not protective. Neither 15dPGJ2 nor troglitazone protected HT-22 cells after the H₂O₂ insult was administered.

The rationale is that if either of the PPAR-γ agonists activates the transcriptional and translational cascade of a neuroprotective protein, or inhibits that of a toxic one, then, a sufficient preincubation time with 15d-PGJ2 or troglitazone should provide enough time for that event to occur. Thus, upon the withdrawal of the medium containing the compound, the neuroprotective cascade stimulated by that compound as a result of preincubation is less likely to be altered because the compound has already mediated its effects. In FIG. 16, HT-22 cells were preincubated with 15d-PGJ2 and troglitazone for 24 hours. Then, the medium containing either of the compounds was washed away, and the new media containing the glutamate insult were added. Cells pre-incubated with 15d-PGJ2 were protected in a dose dependent manner after the withdrawal of the compound- contained-medium. There was a loss of neuroprotection when troglitazone was withdrawn.

### EXAMPLE 6

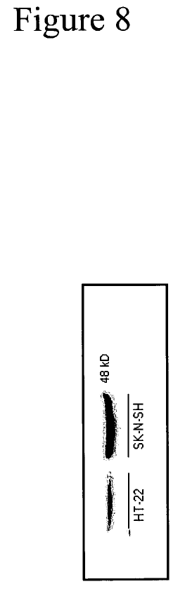

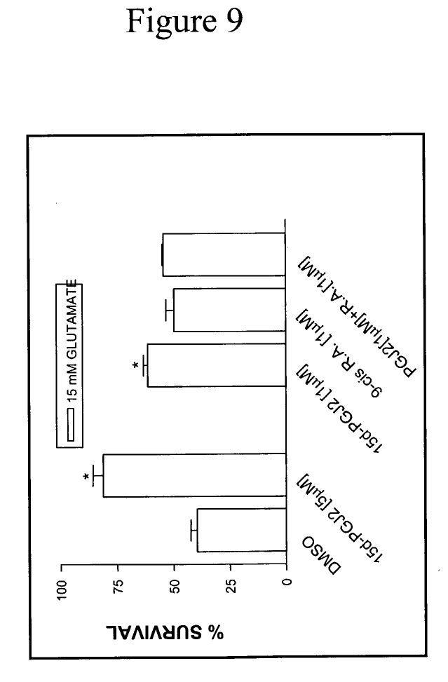

Expression of PPARγ protein in HT-22 and SK—N—SH cytosolic extracts showed a 48 kD band that reacted with an antibody directed at PPARγ (FIG. 8). Additionally, the neuroprotective effects of 15d-PGJ2 and troglitazone were mediated through the PPARγ pathway. By itself, PPAR exhibits a low affinity for DNA; high affinity binding requires heterodimerization with RXR, the 9-cis retinoic acid receptor (Dussault and Forman, 2000). The inventors showed that adding 9-cis retinoic acid did not increase the activity of 15d-PGJ2. As FIG. 9 shows, effects of 15d-PGJ2,9-cis-retinoic acid, and their combination on HT-22 cell viability during glutamate exposure. By adding a 1 μM concentration of 9-cis retinoic acid did not alter the neuroprotective effects of 15d-PGJ2. The protective effect of a 5 mM concentration of 15d-PGJ2 is shown to demonstrate that the lack of additive effect is not due to maximizing neuroprotection at 1 mM. Shown are mean±SEM for n=4 cultures/group. The asterisk (“*”) indicates p<0.05 vs. control (DMSO).

The compounds 15d-PGJ2 and troglitazone both failed to protect either the HT-22 or the SK—N—SH cells against serum deprivation, despite their ability to protect HT-22 cells against glutamate and H₂O₂ insults. This absence of efficacy during serum deprivation may be due to different pathways leading to cell death during serum deprivation versus oxidative insults. The toxicity of glutamate in HT-22 cells is mediated through oxidative stress (Murphy et al., 1989). Glutamate blocks cystine uptake by inhibiting the glutamate/cystine antiporter (Murphy et al., 1989). Since cysteine is required for glutathione (GSH) synthesis, the intracellular concentration of GSH decreases as a consequence. Inasmuch as HT-22 cells lack ionotropic (NMDA) glutamate receptors, the glutamate-induced cell death appears to occur via a slow onset oxidative stress (Li et al., 1997; Maher and Davis, 1996). Morphologically, glutamate treated cells undergo a form of cell death distinct from either necrosis or apoptosis, characterized by plasma membrane blebbing and cell shrinkage, but unlike apoptosis, no DNA fragmentation is observed and the nuclei remain intact (Tan et al., 1998). By contrast, serum deprivation appears to initiate more characteristics of apoptosis (Miller and Johnson, 1996; Tanabe et al. 1998), and is resistant to protection by either 15d-PGJ2 or troglitazone.

The neuron type specificity of the neuroprotective effects of troglitazone is supported by recent reports. Nishijima et al. (2001) have shown that troglitazone improves survival of rat motoneurons against brain-derived neurotrophic factor (“BDNF”) withdrawal, but does not promote the survival of hippocampal neurons. Additionally, neuroprotection by troglitazone has been recently demonstrated in vivo. Sundararajan et al. (2001) reported that troglitazone reduced infarct size and improves functional outcome following cerebral ischemia in rats. In contrast to the present report of neuroprotection with 15d-PGJ2, Rohn et al. (2001) reported that incubation of cortical neurons and SH-SY5Y human neuroblastoma with 10 μM of 15d-PGJ2 induced morphological changes including neurite degeneration and nuclear condensation and fragmentation that were consistent with neurons dying by apoptosis. At this dose of 15d-PGJ2, we observed toxicity in both HT-22 and SK—N—SH cells.

Although not wanting to be bound by theory, despite the distinction of 15d-PGJ2 and troglitazone being PPARγ agonists, the inventors do believe that the PPARγ receptor mediates the neuroprotective effects of these compounds for several reasons. First, both HT-22 and SK—N—SH cells express PPARγ, but SK—N—SH cells are not protected by either troglitazone or 15d-PGJ2. Second, L-783483, which strongly activates PPARγ with an EC50 of 10 nM (Berger et al., 1999), and ciglitazone, another thiazolidinedione, and a PPARγ ligand with similar affinity to 15d-PGJ2 (Lehmann et al., 1995) did not protect HT-22 cells against glutamate cytotoxicity. Third, by itself, PPAR exhibits a low affinity for DNA; high affinity binding requires heterodimerization with RXR, the 9-cis retinoic acid receptor (Benson et al., 2000; Dussault and Forman, 2000). Although not wanting to be bound by theory, examples in the present invention reveal no interaction between 9-cis retinoic acid and 15d-PGJ2 on neuroprotection.

### EXAMPLE 7

After demonstrating that two PPAR-γ ligands were neuroprotective in the initial sets of experiments, the next set of experiments were aimed at determining whether or not the neuroprotective effects were mediated through the PPAR-γ receptor. Although not wanting to be bound by theory, the neuroprotective effects of PPAR-γ ligands appear to be independent of the PPAR receptor. For example, various effects of PPAR-γ ligands that are independent of the PPAR receptor have been reported in several cell types (34-36). Evidence from our experiments supports the hypothesis that the neuroprotective effects of PPAR-γ ligands are related to their structures, and are independent of the PPAR-γ receptor. A specific antagonist (PPAR-γ antagonist) was tested by the neuro-protection of the PPAR-γ ligands. Bisphenol diglycidyl ether (BADGE) is the only specific PPARγ antagonist identified to date. Unfortunately, at concentrations close to its Ki value (100 μM), BADGE was not very soluble and showed some toxicity in our model. Thus, our initial experiments to assess whether the neuroprotective effects of 15d-PGJ2 and troglitazone were PPAR receptor dependent were not conclusive. However, other PPAR-γ agonists were screened for neuroprotection.

### EXAMPLE 8

Assessing γ-glutamylcysteine synthetase (“GCS”) activity was determined as described by Ohno et al. (61, 62), Davis et al. (74), and Oppenheimer et al. (75). GCS activity was determined by measuring the rate of formation of [³H] glutamate-labeled γ-glutamylcysteine. The GCS formed was measured in a reaction mixture (final volume, 50 μl) containing 100 mM Tris-Cl buffer (pH 8.0), 50 mM KCl, 20 mM MgCl₂, 2 mM EDTA, 10 mM ATP, 5 mM cysteine, 5 mM [³H] glutamic acid (0.8mCi/mmol) and the cell extracts (100 μg of soluble proteins). After incubation for 10 min at 37° C., the reaction was terminated by adding 50 μl of acetic acid and 800 μl of acetone, then the mixtures was centrifuged at 15,000×g for 10 min to separate the labeled products and cellular proteins. The supernatants was evaporated to dryness and the residual labeled products was subjected to HPLC analysis. HPLC was performed on a Nucleosil 5NH₂ column (4.6×250 mm) with 50 mM KH₂PO₄-acetonitrile (3:1) acidified by phosphoric acid to pH 3.5 as a mobile phase at a flow rate of 1.0 ml/min and monitored at 230 mm using a Jasco TWINGLE liquid chromatograph. Fractions corresponding to γ-GCS was collected and the radioactivity was measured.

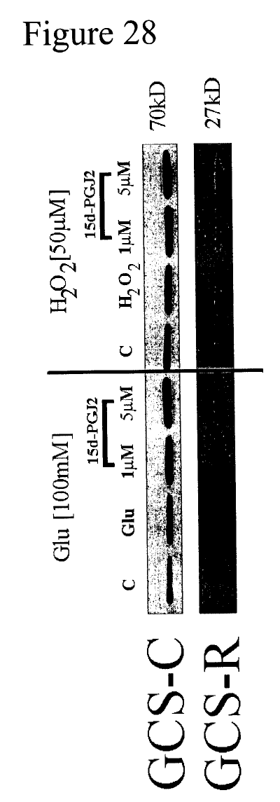

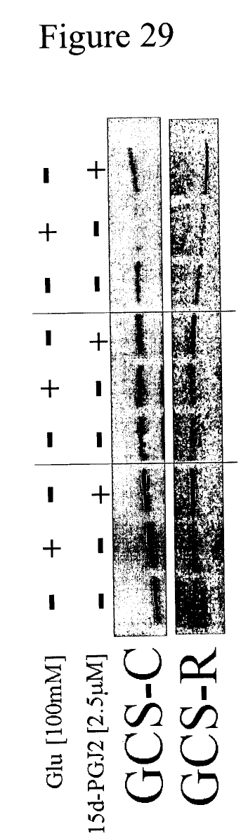

Determining γ-glutamylcysteine synthetase (“GCS”) protein levels was determined using Western blotting analysis, as shown in FIGS. 28 and 29. Antibodies against the heavy (catalytic) and light (regulatory) subunits, GCSH and GCSL, respectively, were ordered from Santa Cruz Biotechnology, Santa Cruz, Calif. Anti-β-actin polyclonal antibody was obtained from Biomedical Technologies Inc. (Stoughton, Mass.). Cyclopentenone PGs treated and untreated HT-22, C6, and RGC-5 cells were homogenized in a buffer containing 20 mM HEPES, pH 7.4, 2 mM EGTA, 1 mM EGTA, 11 mM PMSF, 2 mm DTE, and 101g/ml aprotonin. Cells were sonicated to disrupt cell membranes, and Triton X-100 was added to a final concentration of 0.05% to solubilize membrane bound proteins. Equal amounts of proteins, as determined by the Bradford method, were separated by SDS-PAGE and transferred to polyvinylidene fluoride membrane (Millipore, Bedford, Mass.) in a BiORad (Hercules, Calif.) trans-Blot electrophoresis apparatus at 100 V for 2 hr using Towbin's buffer [25 mm Tris, pH 8.3, 192 mM glycine, and 20% methanol]. The membranes containing immobilized proteins were blocked with 5% skim milk in TS buffer (20 mM Tris, pH 7.5, and 0.5 M NaCl). A polyclonal Anti-GCS was prepared in TS buffer and added to the transblots for overnight incubation. After washes, the membranes were incubated with a goat anti-rabbit IgG HRP conjugated antibody as the secondary antibody in TS buffer. Immunoreactive bands were visualized by a standard ECL procedure.

As shown in FIG. 28, 15d-PGJ2 upregulates the catalytic (C) and regulatory (R) subunits of γ-glutamyl-cysteine synthetase (“GCS”), the rate limiting enzyme in glutathione synthesis. HT-22 cells were plated in 100 mm dishes, and 15d-PGJ2 was added. 24 hours later, glutamate (100 mM) or H₂O₂ (30 μM) were added. About 24 hours post glutamate or H₂O₂ insults, cells were harvested. Equal amounts of proteins, as determined by the Bradford method, were separated by SDS-PAGE and transferred to PVDF membrane. Primary antibodies for GCSC and GCSR were used. A standard Western blotting procedure was carried out, and immunoreactive bands were visualized by ECL procedure.

Similarly, FIG. 29 shows that 15d-PGJ2 upregulates the catalytic (C) and regulatory (R) subunits of γ-glutamyl-cysteine synthetase (“GCS”), the rate limiting enzyme in glutathione synthesis, independent of glutamate. HT-22 cells were plated in 100 mm dishes, and 15d-PGJ2 was added. 24 hours later, glutamate (100 mM) was added. About 24 hours post glutamate insult, cells were harvested. Equal amounts of proteins, as determined by the Bradford method, were separated by SDS-PAGE and transferred to PVDF membrane. Primary antibodies for GCSC and GCSR were used. A standard Western blotting procedure was carried out, and immunoreactive bands were visualized by ECL procedure.

### EXAMPLE 9

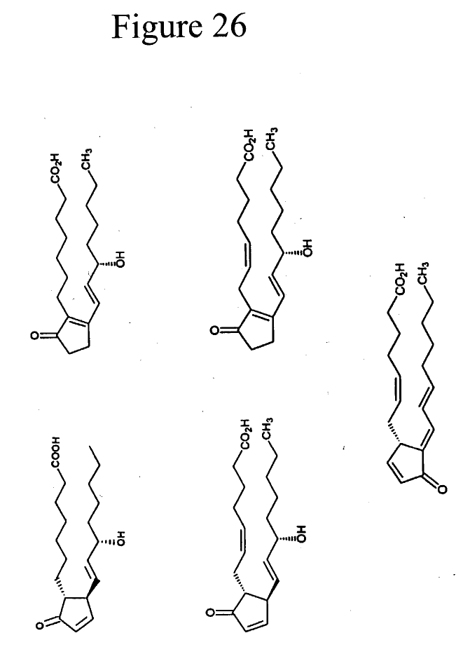

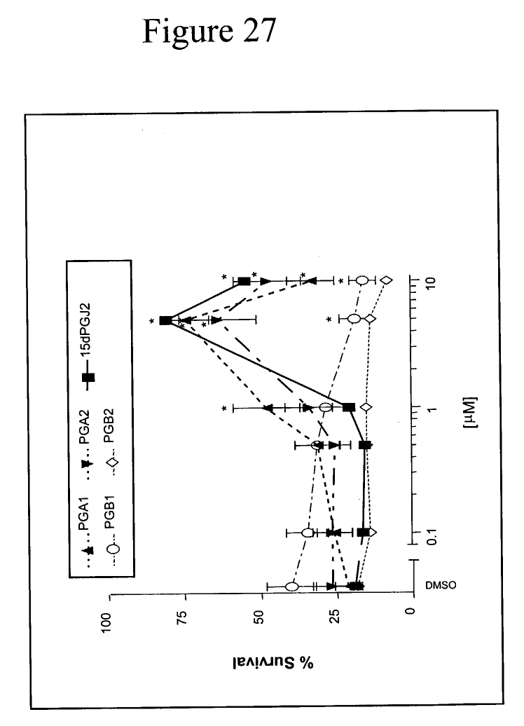

The neuroprotective effects of different chemical structures of prostaglandins (FIG. 26) in HT-22 cells during glutamate exposure are shown in FIG. 27. Although not wanting to be bound by theory, the neuroprotective effects of 15d-PGJ2, PGA1, and PGA2 in HT-22 cells during glutamate exposure are dependent on the reactive unsaturated group of the cyclopentenone ring. For example, HT-22 cells were incubated in 96-well plates, and various concentrations of 15d-PGJ2, PGA1, PGA2, PGB1, and PGB2 were added. Twenty-four hours later, glutamate (15 mM) was added. Fifteen hours following the glutamate insult, cell viability was determined using the calcein AM assay (see FIG. 22), and expressed as % survival of control (non-glutamate-treated cultures). DMSO received the vehicle only for all of the compounds. Shown are mean±SEM of n>6 cultures/group. The asterisk (“*”) indicates p<0.05 vs. respective control groups.

### EXAMPLE 10

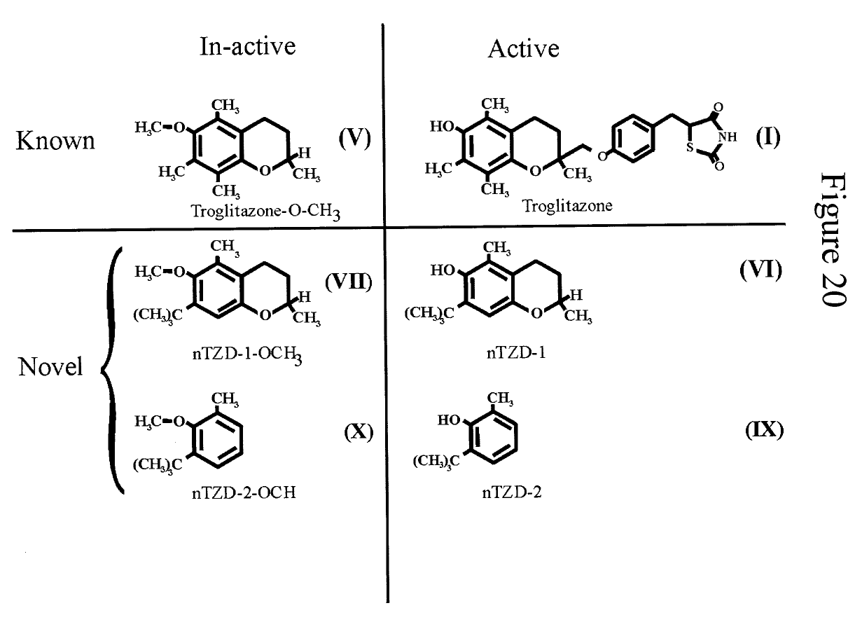

Novel TZD's (“nTZD's”) can be synthesize that possess a phenolic ring structure, as shown in FIG. 20. This novel class of nTZD's will have the advantage of containing a PPAR-γ binding moiety, as well as, a neuroprotective phenolic ring. Moreover, a replacement of the hydrogen (H) of the hydroxyl (OH) group in the vitamin E moiety of troglitazone with a methyl (CH₃) group will synthesize the O-methyl-troglitazone molecule (FIG. 20), the latter compound being devoid of neuroprotective activity. A general formula for neuroprotective phenolic ring structures are shown in the general structural formula (III), and (IV):

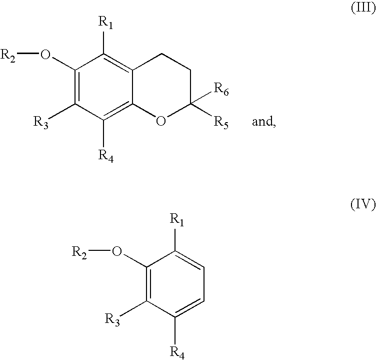

wherein, R₁ is a hydrogen or a methyl group; R₂ is hydrogen; R₃ is hydrogen, methyl group, or tertiary butyl group; R₄ is hydrogen or methyl group; R₅ is hydrogen or methyl group; and R₆ is hydrogen, an alkoxy-benzyl group, or an alkoxy benzyl thiazolidinedion group.

Although not wanting to be bound by theory, TZD's that have a phenolic ring are neuroprotective. To show that the neuroprotective effects of TZD's have a structure-neuroprotective relationship, two TZD's were tested. Both troglitazone and ciglitazone possess a PPAR-γ binding domain, yet troglitazone has a vitamin E, and thus a phenolic ring moiety, whereas, ciglitazone does not. Between troglitazone and ciglitazone only troglitazone exhibited dose dependent neuroprotective effects against glutamate, H₂O₂, and BSO. The affinity of ciglitazone for PPAR-γ is in the low μM range (10). At concentrations up to 40 μM, ciglitazone did not have any neuroprotective effects. Although not wanting to be bound by theory, a phenolic ring may be required for neuroprotection, and has been demonstrated for other compounds beside TZD's. For example, Green and Simpkins (78) have demonstrated that estrogens with an intact phenolic-A ring were neuroprotective. Furthermore, substitution of the hydrogen (H) of the hydroxyl group (OH) in the phenolic ring with a methyl (CH₃) group resulted in the loss of neuroprotection (78). Although not wanting to be bound by theory, the various thiazolidinediones (“TZD's”), and novel synthesized compounds are neuroprotective.

The novel TZD's are first tested in vitro in HT-22, C6, and RGC-5 cell lines against glutamate, H₂O₂, and BSO. Experiments are carried out in a similar fashion to that stated previously. In vitro Neuroprotective nTZD's were assessed for 1) their direct anti-oxidant effects, and 2) their ability to inhibit PKC activity. Rosiglitazone was provided from SmithKline Beecham. Piogliglitazone was provided by Gary Landreth, Ph.D. from Case Western Reserve in Cleveland, Ohio. nTZD's were synthesized as stated earlier.

### EXAMPLE 11

Evaluating the role of cyclopentenone PGs and TZD's has also been completed in vivo. To determine if the cell and insult selectivity of neuroprotection by 15d-PGJ2 and troglitazone in vitro could be demonstrated in vivo, we treated rats with doses of troglitazone, wherein the brain concentrations of about 7 μM were completed, prior to a middle cerebral artery (“MCA”) occlusion. Troglitazone reduced the resulting infarct size by about 50% compared to vehicle (corn oil treated animals). This effective protection from a transient focal ischemic event by troglitazone argues that selective PPARγ agonists may be useful neuroprotectants during neurodegenerative events (e.g. stroke).

Potent neuroprotective cyclopentenone PGs, TZD's, and nTZD's that are protective in cultured neuronal cells, are also protective in animals. To evaluate the neuroprotective role of pre-existing PPAR-γ ligands in vivo, cyclopentenone PGs, and nTZD's were tested in a middle cerebral artery occlusion (“MCAO”) model. Previous studies by Satoh et al. (79) revealed that some cyclopentenone PGs labeled as neurite outgrowth-promoting prostaglandins (“NEPPs”) protected HT-22 against oxidative glutamate, and that NEPP-11 was the most potent compound. In vivo, NEPP-11 protected the brain against a middle cerebral artery occlusion (“MCAO”); intraventricular administration of NEPP-11 (660 ng) 30 min before MCA occlusion decreased the infarct volume by about 50% (79).

As shown below, the results from our experiments comprise results that indicate potent neuroprotective TZD's, as were used in neuronal cell lines are also effective in an animal model. Although not wanting to be bound by theory, the rat is the appropriate animal model for assessing the effects of drugs on neuronal damage, as the compounds to be evaluated are intended for use in humans. Our data shows that a 12h and a 2h subcutaneous (s.c.) injection of troglitazone to ovariectomized female Sprague Dawley rats before the onset of middle cerebral artery occlusion (“MCAO”) reduced the ischemic lesion area by about 50% compared to control animals. Female Sprague Dawley rats were purchased from Harlan in Indianapolis, Ind. Rats were housed in pairs in hanging, stainless steel cages in a temperature-controlled rooms (25±1° C.) with a daily light cycle (on from 0700 to 1900 h daily). All rats were allowed free access to laboratory chow and tap water. All procedures performed on animals will were reviewed and approved by the University of North Texas Health Science Center Institutional Animal Care and Use Committee (“ACUC”).

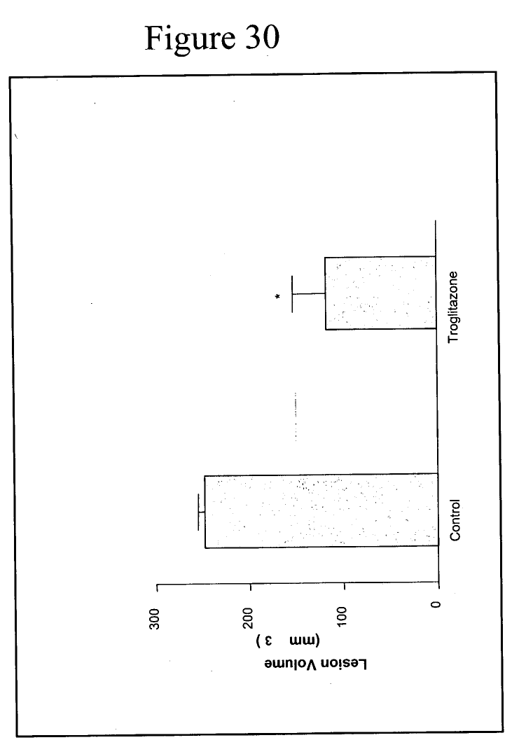

The female Sprague Dawley rats (225-250 g BW) were bilaterally ovariectomized using a dorsal approach. Animals were anesthetized with ketamine (60 m/kg) and xylazine (10 mg/kg). A small (1 cm) cut was made through the skin, fascia, and muscle. The ovaries were externalized, clipped, and removed; then the muscle, fascia, and skin will be sutured closed. Ovariectomy was performed 2 weeks before experiments. As shown in FIG. 30, ovariectomized female Sprague Dawley rats were used to assess the neuroprotective effects of Dawley rats were used to assess the neuroprotective effects of troglitazone in vivo. Either vehicle/control (corn oil) or troglitazone was administered by subcutaneous (s.c.) injection 12 h and 2 h before the onset of MCAO. The middle cerebral artery was occluded for 60 min, and the occluded area was reperfused for 24 hours. Animals were then decapitated, and the brains were removed Animals were then decapitated, and the brains were removed and dissected coronally into 2-mm sections. Percent ischemic lesion area was calculated. * indicates p<0.05 vs. respective control group.

Discussion:

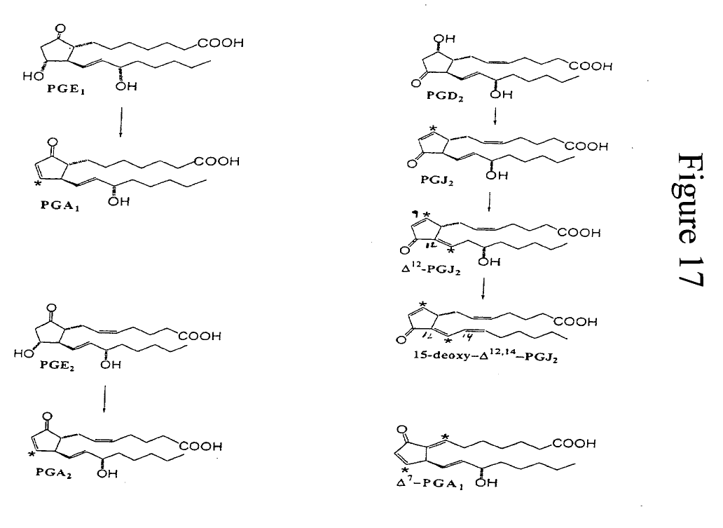

Neuroprotection of the reactive α,β-unsaturated carbonyl group. Although not wanting to be bound by theory, potential mechanisms of neuroprotection by PPAR-γ ligands may include neuroprotective effects of 15d-PGJ2 that are mediated by the reactive α,β-unsaturated carbonyl group of its cyclopentenone ring. Prostaglandins (“PGs”) are a family of biologically active molecules having a diverse range of actions depending upon the PG type and cell target. 15d-PGJ2 is synthesized from arachidonic acid via enzymatic conversion by cyclooxygenase and prostaglandin D₂ (“PGD₂”) synthase, followed by non-enzymatic dehydration of PGD2 (38). The PGA and PGJ series prostaglandins are characterized by the presence of a cyclopentenone ring, which contains a reactive α,β-unsaturated carbonyl group as shown in several illustrations in FIG. 17. The v-unsaturated carbonyl is required for at least some of the biological activities of the cyclopentenone prostaglandins, including the induction of heat shock gene expression (39, 40), the elevation of glutathione levels (41,42), and the inhibition of NF-κB (35,43).

Although not wanting to be bound by theory, 15dPGJ2 may mediate some of its effects through PPAR-γ activation (9,44,45). PPAR independent mechanisms of 15dPGJ2 have also been demonstrated (35,36). Since our data support a PPAR-independent mechanism of neuroprotection, we have investigated the role of the reactive α,β-unsaturated carbonyl group in mediating the neuroprotective effects of 15d-PGJ2 in relation to the: 1) glutathione status within the cell 2) the NF-κB pathway.

As mentioned previously, the reactive α,β-unsaturated carbonyl group is required for some of the biological activities of the cyclopentenone prostaglandins. One of the biological activities of the α,β-unsaturated carbonyl group studied is its relation to glutathione (“GSH”) levels and status within the cell. Ohno et al. (41,42) demonstrated that PGA₂ and 4-hydoxy-2-cyclopentenone induced marked elevation of cellular GSH content in L-121 murine leukemia cells, and this elevation was due to induction of γ-glutamylcysteine synthetase, the rate-limiting enzyme of GSH biosynthesis. The induction of γ-glutamylcysteine synthetase was also found in other cultured mammalian cells such as HeLa S3, NIH/3T3, and porcine aorta endothelial cells (42). Ohno et al. also demonstrated that PGJ₂ as well elevated GSH content in L-1210 cells (42).

Although not wanting to be bound by theory, physiological actions of the E, D, and F series prostaglandins are mediated by binding to specific high-affinity G-protein coupled prostanoid receptors (46). On the other hand, PGs of the A and J series are actively transported into cells by a specific carrier on the cell membrane, and accumulate in cell nuclei with binding to nuclear proteins (47). The α,β-unsaturated carbonyl group of PGA and PGJ series contains an electrophilic center which makes these prostaglandins susceptible to undergoing addition reactions (Michael addition) with nucleophiles such as the free sulfhydryl group of cysteine residues located on cellular proteins (48,49). Once the cyclopentenone PGs get to the nucleus, Narumiya et al. argued that the electrophilic carbons modify the thiol group and regulate the functions of nuclear proteins that are required for gene transcription (50) including that of γ-glutamylcysteine synthetase (42). To further support this hypothesis, Ohno et al. (42) demonstrated that PGs lacking the carbonyl group and the non-substituted double bond in the cyclopentane ring (i.e., PGB2) did not elevate GSH.

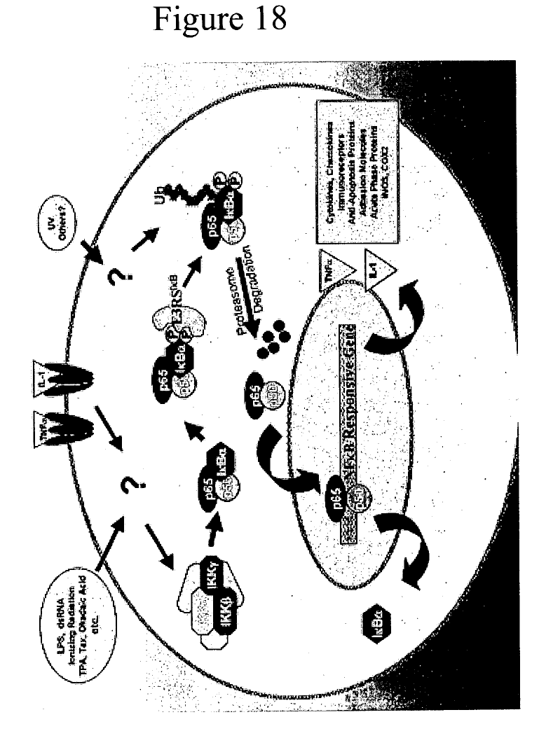

15dPGJ2 and NF-κB: Although not wanting to be bound by theory, NF-κB may be implicated as a major target for PPAR-dependent as well as independent activity of 15d-PGJ2 (35,43). FIG. 18 illustrates the NF-κB pathway. In resting cells, NF-κB is sequestered in the cytoplasm by association with an inhibitory protein IκB. In response to signaling by various factors, IκB kinase (IKK) is activated and phosphorylates IκB on two serine residues. IκB is then ubiquitinated and degraded by the proteasome, freeing NF-κB to migrate into the nucleus and activate gene expression (51). NF-κB plays a role in the transcriptional switch-on of many genes that are activated during the immune response (52), inflammation (53). Furthermore, NF-κB activation has been linked to the pathogenesis of oxidative-stress associated neurodegenerative disorders (21-26).

The role of the reactive α,β-unsaturated carbonyl group in the inhibition of NF-κB activity. The hypothesis that specific cysteine residues in cellular proteins are the key targets for the electrophilic carbons of cyclopentenone prostaglandins has also been studied in relation to NF-κB. Although not wanting to be bound by theory, the 15d-PGJ2 compound represses the activity of NF-κB by targeting different protein cysteine residues (35,43). One of these is located in the activation loop of IκB kinase (IKK), which is required for NF-κB activation (35,43). The target for 15d-PGJ2 in IKK is C179 (35). Alkylation of this cysteine by 15d-PGJ2 evidently interferes with phosphorylation of the activation loop by upstream kinases such as NIK and NAK, thus preventing IKK activation (54). In such, 15d-PGJ2 prevents IκB degradation and nuclear entry of NF-κB.

The other protein cysteine residues are located in the DNA binding domain of NF-κB (43). These cysteine residues are C62 in p50 and C38 in p65, and they are conserved among all Rel proteins (43). Alkylation of these cysteine residues by 15d-PGJ2 results in inhibition of DNA binding by NF-κB. Cemuda-Morollon et al. demonstrated that the p50 subunit is a target for covalent modification by 15d-PGJ2, both in vitro and in intact cells, and that this interaction results in the inhibition of NF-κB binding to DNA (55).

Other cyclopentenone PGs also inhibit NF-κB activation. To further elucidate that the inhibition of NF-κB by PGs is dependent on the presence of a reactive cyclopentenonic moiety, Rossi et al. proved that PGA1 as well inhibited NF-κB activity (56). The authors showed that PGA1 inhibits the phosphorylation and thus the degradation of IκB and therefore prevents the nuclear entry of NF-κB.

Although not wanting to be bound by theory, the NF-κB pathway is regulated by many factors including the redox status of the cell (21). The oxidant status of the cytosol increases the phosphorylation and degradation of IκB, and thus activating NF-κB (21). As mentioned earlier, NF-κB activation has been linked to the pathogenesis of oxidative-stress associated neurodegenerative disorders (21-26), and most stimuli that can induce NF-κB activity are known to induce reactive oxygen species (“ROS”) (22). Three major findings strongly suggest an involvement of NF-κB in Alzheimer's disease (AD). (1) βamyloid (Aβ) can activate NF-κB (23), (2) antioxidants that block activation of NF-κB (24) can protect neurons against oxidative stress-induced cell death (23,25), and (3) two NF-κB DNA binding sites are present in the regulatory region of the amyloid β protein precursor (AβPP) gene (26) which is rapidly induced in response to stress conditions (57).

Oxidative stress is linked to the NF-κB pathway, and the results are detrimental to cell survival. In our cell models, Glutamate and H₂O₂ induce cell death via the oxidative pathway. We demonstrated that 15d-PGJ2 protected, in a dose-dependent manner, HT-22, C6 glioma, and RGC-5 against these oxidative insults. Therefore, we find it highly valuable to explore the role of the NF-κB pathway in the neuroprotective effects of 15d-PGJ2.

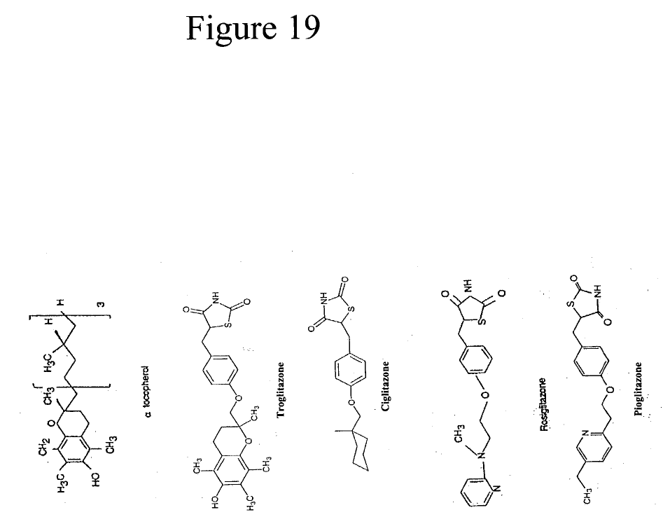

The phenolic ring may be necessary for the neuroprotective effects of TZD's: The thiazolidinediones (“TZD's”) are insulin sensitizer drugs that decrease blood glucose in diabetic animal models (20,58) and in patients with non-insulin-dependent diabetes mellitus (59,60) through alleviating insulin resistance (58). The TZD's bind and activate PPAR-γ (6), and the antidiabetic activity of these compounds is correlated with activation of PPAR-γ (61). Troglitazone belongs to (TZD's) class of drugs. In contrast to other TZD's, troglitazone has a vitamin E moiety (FIG. 19). This provides the compound with potent antioxidant activity (68,69). In vitro, troglitazone prevents lipid peroxidation of LDL (80). In vivo, troglitazone was shown to reduce reactive oxygen species (“ROS”) generation by leukocytes and lipid peroxidation in obese subjects (64).

Another potential effect of troglitazone is the inactivation of PKC activity. The increase in PKC activity is believed to cause diabetic complications such as retinopathy, nephropathy, and neuropathy (65). Troglitazone suppresses hyperglycemia-induced inhibition of insulin receptor tyrosine kinase activity in a manner similar to H7, a PKC inhibitor (66,67), and phorbolester-mediated increase in membrane associated PKC activity in cardiomyocytes (68).

Troglitazone, as already stated, has a vitamin E moiety. Vitamin E is an antioxidant, and further has the ability to inhibit PKC activity (69,70). PKC activation is involved in glutathione depletion and cell death by ROS in C6 glioma cells (71). Our laboratory demonstrated that the inhibition of PKC in HT-22 cells was shown to be protective against oxidative glutamate insult (72). Although not wanting to be bound by theory, these findings suggest that PKC may be a link between oxidative damage and neuronal degeneration. Therefore, troglitazone may improve neuronal survival in our cell models because of its vitamin E moiety that is acting as a direct anti-oxidant and/or inhibiting PKC activation.

Although not wanting to be bound by theory, cyclopentenone prostaglandins that contain a reactive α,β-unsaturated carbonyl group are the neuroprotective component of the compound. The neuroprotective effects of 15d-PGJ2 that are mediated by the reactive α,β-unsaturated carbonyl group of its cyclopentenone ring were tested in cell culture. The rationale behind prostaglandins' (“PGs”) function as intracellular signal mediators in the regulation of a variety of physiological and pathological processes is related to the observation that cyclopentenone prostaglandins are actively transported into cells by a specific carrier on the cell membrane, and accumulate in cell nuclei with binding to nuclear proteins (47).

Methods

The methods used in the above examples should be well understood by one with ordinary skill in the art of molecular biology. However, a more detailed description of the methods uses are described below. For example, PGB2 is formed from PGA2 via two sequential double bond isomerizations (54). The difference between the two compounds is that PGA2 contains a reactive α,β-unsaturated carbonyl group with a reactive electrophilic carbon atom, whereas PGB2 does not. Ohno et al. demonstrated that PGA2 and J2 were observed to elevate the glutathione (“GSH”) content in L-1210 murine leukemia cells, while PGB2 did not. We have observed a potent neuroprotective effect by 15d-PGJ2, and to a lesser extent with PGJ2. However, PGB2 did not display any neuroprotective effects in HT-22 cells against glutamate toxicity.

In order to conduct these experiments, the following compounds: PGA1; PGA2; PGJ2; Δ¹²PGJ2; and 15d-PGJ2 were ordered from Biomol, Plymouth Meeting, Pa. HT-22, an immortalized mouse hippocampal cell line, C6 rat glioma, RGC-5 retinal ganglion cells were used. Cells were grown to confluence in DMEM media, and supplemented with 10% charcoal/dextran-treated fetal bovine serum (“FBS”), and 5 mg/ml gentamycin at 37° C. under 95% air, 5% CO₂. HT-22, C6, and RGC-5 cells were plated at a density of 50,000, 35,000, and 10,000 cells/ml, respectively, in 96-well plates. Wells were pre-treated with cyclopentenone PGs at various times prior to being subjected to glutamate, hydrogen peroxide (H₂O₂), or BSO insults.

The assessment of GSH levels and activity was also evaluated. Cells were suspended in 50 mM Tris buffer, pH 7.4 and homogenized. Monochlorobimane (mCB) was added to a final concentration of 100 mM along with glutathione S-transferase (1 U/ml) with GSH standards treated the same way. The homogenate was then incubated at room temperature for 30 min. The GSH-mCB adduct was measured in a fluorimeter with excitation at 380 nm and emission at 470 nm as described by Kamencic et al. (73).

The mRNA levels of γ-glutamylcysteine synthetase were determined using the reverse-transcription polymerase chain reaction (RT-PCR) analysis. HT-22, C6, and RGC-5 were incubated with cyclopentenone PGs. Cells were then be collected and total RNA was extracted using the RNAzol B reagent (Tel-Test Inc., Friendswood, Tex.) and subjected to cDNA synthesis using AMV reverse transcriptase. The PCR primers for the heavy and light subunits of γ-glutamylcysteine synthetase were requested from Huang and Meister at Cornell University Medical College, New York, N.Y. (76). All the test samples were amplified simultaneously with a particular primer pair as per the annealing temperature of the individual set of primers, using a master mix containing all of the compounds in the PCR, except the target cDNA, or in the case of the control, water.

The activation status of NF-κB was evaluated by Electrophoretic Mobility Shift Assays (“EMSA”). EMSA is a method used for the identification and investigation of DNA binding proteins. It is a powerful tool for the detection of factors binding to specific DNA sequences. The method comprises of three steps: 1) preparation of cytoplasmic and nuclear extracts, 2) DNA binding reaction: the protein in the sample being studied is bound to a radiolabelled DNA fragment in vitro. 2) Electrophoretic separation: the DNA-protein complex is separated from the unbound DNA on a non-denaturing polyacrylamide gel.

Cytoplasmic and nuclear extracts were prepared by taking cells that were incubated with cyclopentenone PGs for a desired amount of time prior to exposure to the oxidative insult. The nuclear and cytoplasmic extracts were then prepared. Briefly, the cells were suspended in 100 μl of buffer C (10 mM HEPES, pH 7.9, 1.5 mM MgCl2, 10 mM KCl, 10% glycerol, 1 mM dithiothreitol, 0.5 mM phenylmethylsulfonyl fluoride) and incubated on ice for 15 min. 3 μl of 10% Nonidet P-40 was added to the suspension and briefly vortexed. Following this, the nuclei was pelleted by centrifugation at low speed. The supernatant (cytoplasmic extract) was collected and stored at −80° C. The nuclear pellet was resuspended in 70 μl of buffer D (20 mM HEPES, pH 7.9, 400 mM NaCl, 1.5 mM MgCl2, 0.2 mM EDTA, 20% glycerol, 1 mM dithiothreitol, 0.5 mM phenylmethylsulfonyl fluoride). The suspension was incubated for 20 min at 4° C. followed by a centrifugation at 8,000 g for 5 min. The supernatant containing the nuclear protein extract was transferred to a fresh microcentrifuge tube and stored at 80° C. Protein concentrations of the cytoplasmic and the nuclear extracts was measured with a detergent-compatible Protein Assay Kit (Bio-Rad), using bovine serum albumin as a standard.

A double-stranded oligonucleotide containing the NF-κB DNA-binding consensus sequence, 5′-AGT TGA GGG GAC TTT CCC AGG C-3′, (Santa Cruz Biotechnology) was used to study the DNA binding activity of NF-κB. Briefly, the double-standard NF-kB oligo's (50 ng) was end labeled with (γ-³²P)-ATP (NEN) using T4 polynucleotide kinase. This labeled probe was then purified by ethanol precipitation. A DNA-binding reaction mixture containing 10 μg cytoplasmic or nuclear extract, 10 mM Tris (pH 7.6), 60 mM NaCl, 1 mM DTT, 4 mM MgCl₂, 1 mM EDTA, 6 fmol of ³²P-labeled oligonucleotide (approximately 20,000 cpm) and 5% glycerol in a total volume of 20 μl was incubate in the presence or absence of excess unlabeled oligo's and the binding reaction was carried out for 20 min at 37° C. For supershift assay, 4 μg of nuclear extract was incubated with 1 μg of antibodies for 30 min at room temperature and analyzed by EMSA. After the binding reaction, the samples was subjected to electrophoresis on a 4% native polyacrylamide gel using 0.25×TBE. The gel was dried and autoradiographed.

Protein extracts from cultured cells were subjected to immunoblot analysis using specific antibodies for IκB, p50, and p65 subunits of NF-κB at 1:500 dilution. Cytoplasmic extracts were used for IκB analysis, whereas nuclear extracts were used to study p65 and p50 subunits of NF-kB. Control blots were run using total cellular extracts and an antibody to GAPDH at 1:1000 dilution. The binding of primary antibodies were detected by using peroxidase labeled appropriate secondary antibodies, which were detected, by using diaminobenzidine as substrate. The antibodies for Western blotting (e.g. p50 subunit of NF-κB, a goat polyclonal IgG; p65, of NF-κB, a rabbit polyclonal IgG; IκB rabbit polyclonal IgG, and GAPDH (chicken anti-rabbit GAPDH immunoaffinity-purified monospecific antibody) were ordered from Santa Cruz Biotechnology, Santa Cruz, Calif.

Cells were incubated with cyclopentenone PGs and fixed in 4% paraformaldehyde for the immunolocalization studies. The immunofluorescence for p65 subunit of NF-κB were done by using a specific antibody against p65 and a fluorescein isothiocyanate-labeled goat anti-rabbit secondary antibody. The immunofluorescent cells were photographed using a Nikon Microphot-FXA photomicroscope.

PGA1, PGA2, and PGJ2 are mono-enone prostaglandins; whereas, 15dPGJ2 is a cross conjugated dienone. Mono-enone PGs contain one electrophilic carbon, whereas 15dPGJ2 has two. As mentioned earlier, electrophilic carbons are easily attacked by nucleophillic SH group-containing molecules such as cysteine residues in proteins as well as glutathione (48). In such a case, cyclopentenone PGs may bind to GSH and lower the intracellular concentration of the latter. However, the concentration of GSH inside the cell is high (48). However, Suzuki et al. (77) demonstrated that the binding of NEPPs to cysteine residue in glutathione is reversible, whereas, binding to target proteins is irreversible. The irreversible, covalent binding to intracellular proteins may elicit the neuronal survival-promoting activities of NEPPs.

Assessment of direct anti-oxidant effects were determined by a Dichlorohydrofluorescein (“DCF”) assay for intracellular peroxides: The DCF assay was usednto quantify intracellular hydroxyl groups in 96 well plates. HT-22, C6, and RGC-5 were incubated with TZD's and nTZD's for various periods of time. Cells were treated for 30 min with various free radical generators (H₂O₂, UV light) by using the dichlorofluorescein (“DCF”) assay, modified for use by a fluorescent microplate reader. The nonfluorescent fluorescin derivatives called dichlorofluorescin (“DCFH”) became DCF and emited fluorescenceafter after being oxidized by various oxidants. One skilled in the art will appreciate that by quantifying the fluorescence, it is possible to quantify free radicals generation.

The membrane lipid peroxidation of TZD's and nTZD's cultured cells were studied by measuring the malonyldialdehyde levels by a colorimetric method involving thiobarbituric acid adduct formation. The GSH levels in light-exposed cells was studied by using the 5,5′-dithiobis(2-nitrobenzoic acid) reagent.

PKC is activated by its translocation from the cytoplasm to the plasma membrane, and was determine by measuring the translocation status of PKC and its levels in the cytoplasmic as well as membrane fractions in HT-22, C6, and RGC-5 cells. The separation of cytoplasmic and membrane fractions were performed and anti-PKC polyclonal antibodies were ordered from Santa Cruz Biotechnology, Santa Cruz, Calif. Anti-β-actin polyclonal antibody was obtained from Biomedical Technologies Inc. (Stoughton, Mass.). Cells were homogenized. Cells were sonicated to disrupt cell membranes and the soluble (cytoplasmic) and pellet fractions were separated by centrifugation. The homogenate was centrifuged at 100,000×g, and the soluble (cytoplasmic) fraction was collected. Samples were adjusted by addition or dilution to 0.05% Triton X-100. The pellet (cytoskeletal and lipid materials) was diluted to 0.1% Triton. The homogenate was centrifuged, and the supernatant (membrane protein) was collected. The supernatant was adjusted by dilution in homogenization buffer.

Equal amounts of proteins, as determined by the Bradford method, were separated by SDS-PAGE. The proteins were then transferred to polyvinylidene fluoride membrane (Millipore, Bedford, Mass.) in a BiORad (Hercules, Calif.) trans-Blot electrophoresis apparatus at 100 V for 2 hr using Towbin's buffer. The membranes containing immobilized proteins were blocked with 5% skim milk in TS buffer. A polyclonal Anti-PKC was prepared in TS buffer and added to the transblots for overnight incubation. After several washes, the membranes were incubated with a goat anti-rabbit IgG HRP conjugated antibody as the secondary antibody in TS buffer. Immunoreactive bands was visualized by a standard ECL procedure. A person skilled in the art of molecular biology would be familiar with different variations of Western blot protocols.

The potent neuroprotective cyclopentenone PGs, TZD's, and nTZD's that were used in the above in vitro experiments, were solubilized in corn oil as a vehicle for injection. Either vehicle or the compound was administered by subcutaneous (s.c.) injection, 12 h and 2 h before the onset of MCA occlusion. Animals were anesthetized by intraperitoneal (“ip”) injection of ketamine (60 m/kg) and xylazine (10 mg/kg). Briefly, the left common carotid artery, external carotid artery, and internal carotid artery was exposed through a midline cervical incision. A 3-0 monofilament suture will be introduced into the internal carotid artery lumen and gently advanced until resistance is felt. The suture will be kept in place for 60 min and then withdrawn to allow MCA reperfusion. The procedure will be performed within 20 min, with minimal bleeding. Rectal temperature will be maintained between 36.5 and 37.0 C during the entire procedure.

Each group of animals was decapitated 24 h after MCAO. The brain was then removed and dissected coronally into 2-mm sections using a metallic brain matrix (Harvard). The sections located 3, 5, 7, 9, and 11 mm posterior to the tip of the olfactory bulb were stained by incubation in a 2% solution of 2,3,5-triphenyltetrazolium chloride in a 0.9% saline solution at 37C for 30 min. Slices were then fixed in a 10% formalin and photographed by a digital camera (Sony MVC-FD5), and the ischemic lesion area were determined for each slice using the Image-Pro Plus software (Media Cybernectics, Silver Spring, Md.). Percent ischemic lesion area was calculated as the sum of the ischemic lesion area for the five slices divided by the total cross sectional area of these five slices.

The WY-14643, Ciglitazone, Troglitazone, 9-cis retinoic acid, and 15d-PGJ2 compounds were purchased from Biomol (Plymouth Meeting, Pa.). L-165041 and L-783483 were kindly provided by Merck Laboratories. The cell lines used were HT-22, an immortalized mouse hippocampal cell line, and SK—N—SH, a human neuroblastoma cell line. The HT-22 cells were obtained from David Schubert (Salk Institute, San Diego, Calif.). The HT-22 line was originally selected from HT-4 cells based on glutamate sensitivity. HT-4 cells were immortalized from primary hippocampal neurons using a temperature-sensitive SV-40 T antigen (Morimoto and Koshland, 1990). SK—N—SH were obtained from ATCC (Manassas, Va.). HT-22 and SK—N—SH cells were grown to confluence in DMEM and RPMI-1640 media, respectively, and supplemented with 10% charcoal/dextran-treated fetal bovine serum (FBS), and 5 mg/ml gentamycin at 37° C. under 95% air, 5% CO₂. HT-22 cells were plated at a density of 50,000 cells/ml (5,000 cells/well), and SK—N—SH were plated at a density of 120,000 to 150,000 cells/ml (12,000 to 15,000 cells/well) in 96-well plates. In most studies, wells were pre-treated with PPAR ligands over a wide dose range at various times prior to being subjected to either glutamate, hydrogen peroxide (H₂O₂), or serum deprivation insults. In some studies, the insults were applied prior to the addition of the PPAR ligand.

The cell viability assays were conducted about 14 to 24 hours post-insult time, viability of cells was determined using 2.5 μM Calcein AM assay in phosphate-buffered saline (PBS). After 25 minutes of incubation, live cells were distinguished by the presence of intracellular esterase activity, which cleaves the calcein AM dye, producing a bright green fluorescence. Viability was measured in Relative Fluorescent Units (“RFU”), and expressed as percentage of vehicle-treated control values. The calcein assay was designed to produce results that accurately reflect viable cell numbers. At cell concentrations below 10,000 cells per well (96-well plate) a linear relationship between RFU and cell number is achieved (r²=0.9997, data not shown).

The Western blotting of harvested SK—N—SH and HT-22 cells were homogenized in a buffer containing 20 mM HEPES, pH 7.4, 2 mM EGTA, 1 MM EGTA, 1 mM PMSF, 2 mM DTE, and 10 μg/ml aprotonin. Cells were sonicated to disrupt cell membranes, and the soluble (cytoplasmic) and pellet fractions were separated by centrifugation. The homogenate was centrifuged at 100,000×g, and the soluble fraction was collected. Samples were adjusted by addition or dilution to 0.05% Triton X-100. Equal amounts of proteins, as determined by the Bradford method, were separated by SDS-PAGE and transferred to polyvinylidene fluoride membrane (Millipore, Bedford, Mass.) in a BioRad (Hercules, Calif.) trans-Blot electrophoresis apparatus at 100 V for 2 hr using Towbin's buffer [25 mM Tris, pH 8.3, 192 mM glycine, and 20% methanol]. The membranes containing immobilized proteins were blocked with 5% skim milk in TS buffer (20 mM Tris, pH 7.5, and 0.5 M NaCl). A polyclonal PPARγ (Santa Cruz Biotechnology, Santa Cruz, C.A.) that cross reacts with both human and mouse PPAR-γ was prepared in TS buffer and added to the transblots for overnight incubation. After washes, the membranes were incubated with a goat anti-rabbit IgG HRP conjugated antibody as the secondary antibody in TS buffer. Immunoreactive bands were visualized by a standard ECL procedure.

The statistical analysis was in most experiments was determined by one-way analysis of variance (“ANOVA”) followed by a Tukey's multiple comparison test. P<0.05 was considered significant for all experiments. The values are reported as the mean±SEM.

One skilled in the art readily appreciates that the disclosed invention is well adapted to carry out the objectives and obtain the ends and advantages mentioned as well as those inherent therein. The invention described herein represents the first demonstration of a protective effect of PPAR-γ ligands against oxidative stress in a neuronal cell or animal. The importance of the inventors work lies in this initial discovery, and in showing that 15d-PGJ2 and troglitazone displayed different properties in their neuroprotective effects. Although not wanting to be bound by theory, evidence from these experiments suggest that even though 15d-PGJ2 and troglitazone are both PPAR-γ ligands, their neuroprotective effects may be mediated through a novel pathway(s) independent of the PPAR receptor.

### References Cited

The following U.S. patent documents and publications are incorporated by reference herein.

### U.S. patent Documents

U.S. Pat. No. 6,028,109 Feb. 22, 2000 and entitled “Use of agonists of the peroxisome proliferator activated receptor alpha for treating obesity” with Willson, et al. listed as inventors.

U.S. Pat. No. 5,994,554 filed on Nov. 30, 1999 and entitled “Activators of the nuclear orphan receptor peroxisome proliferator-activated receptor gamma” with Kliewer, et al. listed as inventors.

U.S. Pat. No. 5,902,726 filed on May 11, 1999 and entitled “Activators of the nuclear orphan receptor peroxisome proliferator-activated receptor gamma” with Kliewer, et al. listed as inventors.

U.S. Pat. No. 5,861,274 Jan. 19, 1999 and entitled “Nucleic acids encoding peroxisome proliferator-activated receptor” with Evans, et al. listed as inventors.

### OTHER REFERENCES

Benson, S., Padmanabhan, S., Kurtz, T. W., Pershadsingh, H. A., 2000. Ligands for the peroxisome proliferator-activated receptor-gamma and the retinoid X receptor-alpha exert synergistic antiproliferative effects on human coronary artery smooth muscle cells. Mol. Cell. Biol. Res. Commun. 3(3), 159-164.

Berger, J., Leibowitz, M. D., Doebber, T. W., Elbrecht, A., Zhang, B., Zhou, G., Biswas, C., Cullinan, C. A., Hayes, N. S., Li, Y., Tanen, M., Ventre, J., Wu, M. S., Berger, G. D., Mosley, R., Marquis, R., Santini, C., Sahoo, S. P., Tolman, R. L., Smith, R. G., Moller D. E., 1999. Novel peroxisome proliferator-activated receptor (PPAR)_{y} and PPARδ ligands produce distinct biological effects. J. Biol. Chem. 274(10), 6718-6725.

Chawla, A., Schwartz, E. J., Dimaculangan, D. D., Lazar, M. A., 1994. Peroxisome proliferator-activated receptor (PPAR) y: adipose-predominant expression and induction early in adipose differentiation. Endocrinology 135, 798-800.

Cherubini, A., Polidori, M. C., Bregnocchi, M., Pezzuto, S., Cecchetti, R., Ingegni, T., Di lorio, A., Senin, U., Mecocci, P., 2000. Antioxidant profile and early outcome in stroke patients. Stroke 31, 2295-2300.

Dreyer, C., Krey, G., Keller, H., Givel, F., Helfetbein, G., Wahli, W., 1992. Control of the peroxisomal β-oxidation pathway by a novel family of nuclear hormone receptors. Cell 68, 879-887.

Dubey, R. K., Zhang, H. Y., Reddy, S. R., Boegehold, M. A., Kotchen, T. A., 1993. Pioglitazone attenuates hypertension and inhibits growth of renal arteriolar smooth muscle in rats. Am. J. physiol. 265, R726-R732.

Dussault, I., Forman, B. M., 2000. Prostaglandins and fatty acids regulate transcriptional signaling via the peroxisome proliferator activated receptor nuclear receptors. Prostaglandins & other Lipid Mediators 62, 1-13.

Issemann, I., Green, S., 1990. Activation of a member of the steroid hormone receptor superfamily by peroxisome proliferators. Nature 347, 645-650.

Jenner, P., Olanow, C. W., 1996. Oxidative stress and the pathogenesis of Parkinson's disease. Neurology 47, S161-170.

Kliewer, S. A., Lenhard, J. M., Willson, T. M., Patel, I., Morris, D. C., Lehmann, J. M., 1995. A prostaglandin J2 metabolite binds peroxisome proliferator-activated receptor gamma and promotes adipocyte differentiation Cell 83, 813-819.

Krey, G., Braissant, O., L'Horest, F., Kalkhoven, E., Perroud, M., Parker, M. G., Wahli, W., 1997 Fatty acids, eicosanoids, and hypolipidemic agents identified as ligands of peroxisome proliferator-activated receptors by coactivator-dependent receptor ligand assay. Mol. Endocrinol. 11, 779-791

Law, R. E., Meehan, W. P., Xi, X. P., Graf, K., Wuthrich, D. A., Coats, W., Faxon, D., Hsuch, W. A., 1996. Troglitazone inhibits vascular smooth muscle cell growth and intimal hyperplasia. J. Clin. Invest. 98, 1897-1905.

Lehmann, J. M., Moore, L. B., Smith-Oliver, T. A., Wilkinson, W. O., Willson, T. M., Kliewer, S. A., 1995. An antidiabetic thiazolidinedione is a high affinity ligand for peroxisome proliferator-activated receptor gamma (PPAR gamma). J. Biol. Chem. 270 (10), 12953-12956.

Lemberger, T., Braissant, O., Juge-Aubry, C., Keller, H., Saladin, Regis., Steals, B., Auwerx, J., Burger, A., Meier, C. A., and Wahli, W., 1996. PPAR tissue distribution and interactions with other hormone-signaling pathways. Ann. Of The New York Academy of Sciences 804, 231-251.

Li, Y., Maher, P., Schubert, D., (1997) A role of 12-lipoxygenase in nerve cell death caused by glutathione depletion. Neuron 19, 453-463.

Maher, P., Davis, J. B., (1996) The role of monoamine metabolism in oxidative glutamate toxicity. J. Neurosci. 16, 6394-6401.

Markesbery, W. R., 1997. Oxidative stress hypothesis in Alzheimer's disease. Free Radic. Biol. Med. 23,134-147.

Miller, T. M., Johnson, E. M., Jr., 1996. Metabolic and genetic analyses of apoptosis in potassium/serum-deprived rat cerebellar granule cells. J. Neuroscience 16(23), 7487-7495.

Morimoto, B. H., Koshland, D. E., Jr., 1990. Induction and expresssion of long- and short-term neurosecretory potentiation in a neural cell line. Neuron 5, 875-880.

Murphy, T. H., Miyamoto, M., Sastre, A., Schnaar, R. L., Coyle, J. T., 1989. Glutamate toxicity in a neuronal cell line involves inhibition of cystine transport leading to oxidative stress. Neuron 2, 1547-1558.

Nishijima, C., Kimoto, K., Arakawa, Y., 2001. Survival activity of troglitazone in rat motoneurones. J. Neurochem. 76, 382-390.

Rohn, T. T., Wong, S. M., Cotman, C. W., Cribbs, D. H., 2001. 15-Deoxy-Δ^{12,14}-prostaglandin J2, a specific ligand for peroxisome proliferator-activated receptor-γ, induces neuronal apoptosis. Mol. Neurosc. 12(4), 839-843.

Rudich, A., Tirosh, A., Potashnik, R., Khamaisi, M., Bashan, N., 1999. Lipoic acid protects against oxidative stress induced impairment in insulin stimulation of protein kinase B and glucose transport in 3T3-L1 adipocytes. Diabetologia 42, 949-957.

Sundararajan, S., Wandery, E., Lust, W. D., Landreth, G. E., 2001. A PPARγ agonist reduces infarct size and improves functional outcome following focal ischemia. Soc. Neurosci. Abstract 864.4.

Tan, S., Sagara, Y., Liu, Y., Maher, P., Schubert, D., 1998. The regulation of reactive oxygen species production during programmed cell death. J. Cell Biol. 141(6), 1423-32.

Tan, S., Wood, M., Maher, P., 1998. Oxidative stress induces a form of programmed cell death with characteristics of both apoptosis and necrosis in neuronal cells. J. Neurochem. 71, 95-105.

Tanabe, H., Eguchi, Y., Shimizu, S., Martinou, J-C., Tsujimoto, Y., 1998. Death-signaling cascade in mouse cerebellar granule neurons. Eur. J. Neurosci. 10, 1403-1411.

Tontonoz, P., Hu, E., Spiegelman, B. M., 1994. Stimulation of adipogenesis in fibroblasts by PPARγ2, a lipid-activated transcription factor. Cell 79, 124-130.

### From Grant

Issemann, I. & Green, S. (1990) Activation of a member of the steroid hormone receptor superfamily by peroxisome proliferators. Nature 347(6294): 645-650.

Lemberger, T., Braissant, O., Juge-Aubry, C., Keller, H., Saladin, Regis., Steals, B., Auwerx, J., Burger, A., Meier, C. A., and Wahli, W. (1996) PPAR tissue distribution and interactions with other hormone-signaling pathways. Ann. Of The New York Academy of Sciences 804:231-251.

Dreyer, C., Krey, G., Keller, H., Givel, F., Helfetbein, G., & Wahli, W. (1992) Control of the Peroxisomal β-oxidation Pathway by a Novel Family of Nuclear Hormone Receptors. Cell 68, 879-887.

Chawla, A., E. J., Schwartz, D. D. Dimaculangan & M. A. Lazar. (1994) Peroxisome Proliferator-Activated Receptor (PPAR) γ: adipose-predominant expression and induction early in adipose differentiation. Endocrinology 135, 798-800.

Tontonoz, P., E. Hu & B. M. Spiegelman. (1994) Stimulation of adipogenesis in fibroblasts by PPARγ2, a lipid-activated transcription factor. Cell 79, 124-130.

Lehmann J. M., Moore, L. B., Smith-Oliver, T. A., Wilkinson, W. O., Willson, T. M., and Kliewer, S. A. (1995) An antidiabetic thiazolidinedione is a high affinity ligand for peroxisome proliferator-activated receptor gamma (PPAR gamma). J. Biol. Chem. 270 (10), 12953-12956.

Dubey R. K., Zhang H. Y., Reddy S. R., Boegehold M. A., Kotchen T. A. (1993) pioglitazone attenuates hypertension and inhibits growth of renal arteriolar smooth muscle in rats. Am. J. physiol. 265: R726-R732.

Law R. E., Meehan W. P., Xi X. P., graf K., Wuthrich D. A., Coats W., Faxon D., Hsuch W. A. (1996) Troglitazone inhibits vascular smooth muscle cell growth and intimal hyperplasia. J. Clin. Invest. 98:1897-1905.

Kliewer S. A., Lenhard J. M., Willson T. M., Patel I., Morris D. C., Lehmann J. M. (1995) A prostaglandin J2 metabolite binds peroxisome proliferator-activated receptor gamma and promotes adipocyte differentiation. Cell 83:813-819.

Dreyer, C., Krey G., Keller H., Givel F., Helftenbein G., and Wahli W. (1992) Control of the peroxisomal beta-oxidation pathway by a novel family of nuclear hormone receptors. Cell 68:879-887 Dussault, I., and Forman, B. M. (2000) Prostaglandins and fatty acids regulate transcriptional signaling via the peroxisome proliferator activated receptor nuclear receptors. Prostaglandins & other Lipid Mediators 62, 1-13.

Wahli W., Braissant O., Desvergne B. (1995) Peroxisome proliferator-activated receptors: transcriptional regulators of adipogenesis, lipid metabolism and more. Chem & Biol. 2:261-266

Krey, G., Braissant, O., L'Horest, F., Kalkhoven, E., Perroud, M., Parker, M. G., and Wahli W. (1997) Fatty acids, eicosanoids, and hypolipidemic agents identified as ligands of peroxisome proliferator-activated receptors by coactivator-dependent receptor ligand assay. Mol. Endocrinol. 11, 779-791

Zinman, B. (2001) PPARγ agonists in type 2 diabetes: how far have we come in ‘preventing the inevitable’? A review of the metabolic effects of rosiglitazone. Diabetes, obesity and Metabolism, 3 (Suppl. 1):S34-S43

Jones N. P., Mather R., Owen S., Porter L. E., Patwardhan R. (2000) Long-term efficacy of rosiglitazone as monotherapy or in combination with metformin. Diabetologia 42(Suppl.1): A192

Garvey W. T., Herrnayer K. L. (1998) Clinical implications of the insulin resistance syndrome. Clin Cornerstone 1:13-28

Suzuki, Y. S., Forman, H. J., Sevanian A. (1997) Oxidants as stimulators of signal transduction. Free radical Biol. Med. 22:269-285

Halliwell B., Gutteridge J. M. C. (1989) Free Radicals in Biology and Medicine. (Clarendon Press, Oxford, ed. 2): 1-81.

Coyle J., Puttfarcken P. (1993) Oxidative stress, glutamate, and neurodegenerative disorders. Science. 262: 689-695.

Fujita T., Sugiyama Y., Taketomi S., Shoda T., Kawamatsu Y., Iwatsuka Y. and Suzuoki Z. (1983) Reduction of insulin resistance in obese and/or diabetic animals by 5-[4-(1-methylcyclohexylmethoxy)benzyl]-thiazolidine-2,4-dione (ADD-3878, U-63,287, ciglitazone), a new antidiabetic agent. Diabetes 32:804-810.

Grilli M. (1996) Neuroprotection by aspirin and sodium salicylate through blockade of NF-kB activation. Science 274:1383-1385.

Lezoualch F., Sagara Y., Holsboer F., Behl C. (1998) High constitutive NF-κB activity mediates resistance to oxidative stress in neuronal cells. Journal of Neuroscience 18(9):3224-3232.

Behl C., Davis J. B., Lesley R., Schubert D. (1994) Hydrogen peroxide mediates amyloid β-protein toxicity. Cell 77:817-827

Schreck R., Zorbas H., Winnacker E. L., Bauerle P. A. (1991) Reactive oxygen intermediates as apparently widely used messengers in the activation of the NF-B transcription factor and HIV-1. EMBO J. 10:2247-2258.

Kaltschmidt B., Uherek M., Volk B., Bacuerle P. A., Kaltschmidt C. (1997) Transcription factor NF-k-B is activated in primary neurons by amyloid beta peptides and in neurons surrounding early plaques from patients with Alzheimer disease. Proc Natl Acad. Sci. 94:2642-2647.

Grilli M, Ribola M, Alberici A, Valerio A, Memo M, Spano P. (1995) Identification and characterization of akB/Rel binding site in the regulatory region of the APP gene. J. Biol. Chem. 270:26774-26777.

Jenner P., Olanow C. W. Pathological evidence for oxidative stress in Parkinson's disease and related degenerative disorders. In: Olanow C. W., Jenner P., Youdim M., eds. Neurodegeneration and neuroprotection in Parkinson's disease. London: Academic 24-45.

Cherubini A., Polidori M. C., Bregnocchi M., Pezzuto S., Cecchetti R., Ingegni T., Di lorio A., Senin U., Mecocci P. (2000) Antioxidant profile and early outcome in stroke patients. Stroke 31:2295-2300.

Nishijima C., Kimoto, K. and Arakawa, Y. (2001) Survival activity of troglitazone in rat motoneurones. J. Neurochem. 76, 382-390.

Heneka M. T., Feinstein D. L., Galea E., Gleichmann M., Wullner U., Klockgether T. (1999) Peroxisome proliferator-activated receptor gamma agonists protect cerebellar granule cells from cytokine-induced apoptotic cell death by inhibition of inducible nitric oxide synthase. J Neuroimmunol. 100(1-2):156-168.

Combs C. K., Johnson D. E., Karlo J. C., Cannady S. B., Landreth G. E. (2000) Inflammatory mechanisms in Alzheimer's disease: inhibition of beta-amyloid-stimulated proinflammatory responses and neurotoxicity by PPAR gamma agonists. J. Neurosci. 20(2):558-567.

Murphy, T. H., Miyamoto M., Sastre A., Schnaar R. L., Coyle, J. T. (1989) Glutamate toxicity in a neuronal cell line involves inhibition of cystine transport leading to oxidative stress. Neuron 2:1547-1558.

Kato S., Negishi K., Mawatari K., Kuo C. H. (1992) A mechanism for glutamate toxicity in the C6 glioma cells involving inhibition of cystine uptake leading to glutathione depletion. Neuroscience 48(4):903-914.

Li, Y., Maher, P., Schubert, D., (1997) A role of 12-lipoxygenase in nerve cell death caused by glutathione depletion. Neuron 19, 453-463.

Maher, P., Davis, J. B., (1996) The role of monoamine metabolism in oxidative glutamate toxicity. J. Neurosci. 16, 6394-6401.

Wilmer W. A., Dixon C., Lu L., Hilbelink T., Rovin B. H. (2001) A cyclopentenone prostaglandin activates mesangial MAP kinase independently of PPAR gamma. Biochem Biophys Res Commun. 281(1):57-62.

Tan S., Wood, M., and Maher, P. (1998). Oxidative stress induces a form of programmed cell death with characteristics of both apoptosis and necrosis in neuronal cells. J. Neurochem. 71, 95-105.

Ito S., Narumiya S., Hayaishi O. (1989) Prostaglandin D2: A biochemical perspective. Prostaglandins, Leukotrenes Essent. Fatty Acids 37:219-234.

Fukushima M. (1990) Prostaglandin J2-antitumor and anti-viral activities and mechanisms involved. Eicosanoids 3:189-199.

Rossi A. Elia G., Santoro M. G. (1996) 2-Cyclopenten-1-one, a new inducer of heat shock protein 70 with anti-viral activity. J. Biol. Chem. 271:32192-32196.

Ohno K., Hirata, m. (1990) Induction of γ-glutamylcysteine synthetase by prostaglandin A2 in 1-1210 cells. Biochem. Biophys. Res. Commun. 168:551-557.

Ohno K., Higaki J., takechi S., Hirata M. (1990) Specific role of an α,β-unsaturated carbonyl group in γ-glutamylcysteine synthetase induction by prostaglandin A2. Chem. Biol. Interactions. 76:77-87

Straus S. D., Pascual G., Li M., Welch J. S., Ricote m., Hsiang C-H, Sengchanthalansy L. L., Ghosh G., Glass C. K. (1999) 15-Deoxy-Δ^{12,14}-prostaglandin J2 inhibits multiple steps in the NF-κB signaling pathway. Proceed. Nat. Acad. Sciences. 97(9):4844-4849.

Jiang C., Ting A. T., Seed B. (1998) PPAR-gamma agonists inhibit production of monocyte inflammatory cytokines. Nature 391:82-86.

Ricote M. Li A. C., Willson T. M., Kelly C. J., Glass C. K. (1998) Nature. 391:79-82.

Narumiya S. (1995) Structures, properties and distribution of prostanoid receptors. Adv prostaglandin Trombox Leukotriene Res. 23:17-22.

Narumiya S., Fukushima M. (1986) Site and mechanism of growth inhibition by prostaglandinds. I. Active transport and intracellular accumulation of cyclopentenone prostaglandins, a reaction leading to growth inhibition. J. Pharmacol. Exp. Ther. 239:500-505

Atsmon J., Sweetman B. J., Baertschi S. W., Harris T. M., Roberts II L. J. Formation of thiol conjugates of 9-deoxy-Δ9Δ12(E)-prostaglandin D2 and Δ12(E)-prostaglandin D2. Biochemistry 29:3760-37665.

Honn K. V., Mamett L. J. (1985) Requirement of a reactive α,β-unsaturated carbonyl for inhibition of tumor growth and induction of differentiation by “A” series prostaglandins. Biochem BiophyRes Commun. 129:34-40.

Narumiya S., Ohno K., Fukushima M., Fujiwara M. (1987) Site and mechanism of growth inhibition by prostaglandins. III. Distribution and binding of prostaglandin A2 and delta 12-prostaglandin J2 in nuclei. J. Pharmacol. Exp. Therap. 242:306-311.

Karin M., Ben-Neriah Y. (2000) Phosphorylation meets ubiquitination: the control of NF-κB activity. Ann Rev Immunol. 18:621-663.

Ghosh S., May M. J., Kopp E. B. (1998) NF-kB and rel proteins: Evolutionarily conserved mediators of immune responses. Ann. Rev. Immunol. 16:225-260.

Bauerele P. A. (1998) IkB-MF-kB structures: at the interface of inflammation control. Cell. 95:729-731.

Straus D. S., Glass C. K. (2001) Cyclopentenone prostaglandins: new insights on biological activities and cellular targets. Medical Research Reviews 21(3): 185-210.

Cemuda-Morollon E, Pineda-Molina E, Canada F J, Perez-Sala D. (2001) 15-Deoxy-Delta 12,14-prostaglandin J2 inhibition of NF-kappaB-DNA binding through covalent modification of the p50 subunit. J. Biol. Chem. 276(38):35530-35536.

Rossi A., Elia G., Santoro M. G. Inhibition of nuclear factor kappa B by prostaglandin A1: an effect associated with heat shock transcription factor activation. Proc Natl Acad. Sci. 94(2):746-750.

Siman R, Card J P, Nelson R B, Davis L G (1989) Expression of β[Y. Wenl]-amyloid precursor protein in reactive astrocytes following neuronal damage. Neuron 3:275-285.

Saltiel A. R. and Olefsky J. M. (1996) Thiazolidinediones in the treatment of insulin resistance and type II diabetes. Diabetes 45:1661-1669.

Iwamoto Y., Kuzuya T., Matsuda A., Awata T., Kumakura S., Inooka G., Shiraishi, I. (1991) Effect of new oral antidiabetic agent CS-045 on glucose tolerance and insulin secretion in patients with NIDDM. Diabetes Care 14:1083-1086.

Suter S. L., Nolan J. J., Wallace P., Gumbiner B., Olefsky J. M. (1992) Metabolic effects of new oral hypoglycemic agent CS-045 in NIDDM subjects. Diabetes Care 15:193-203.

Wilson T. M., Cobb J. E., Cowan D. J., Wiethe R. W., Correra I. D., Prakash S. R., Beck K. D., Moore L. B., Kliever S. A., Lehmann J. M. (1996) The Structure-Activity Relationship between Peroxisome Proliferator-Activated Receptor Agonism and the Antihyperglycemic Activity of Thiazolidinediones. J.Med.Chem. 39(3):665-668.

Noguchi N., Sakai H., Kato Y., Tsuchiya J., Yamamoto Y., Niki E., Horikoshi H., Kodama T. (1996) Inhibition of oxidation of low density lipoprotein by troglitazone. Atherosclerosis 123:227-234.

Cominacini L., Garbin U., Pastorino A. M., Campagnola M., Fratta-Pasini A., Davoli A., Rigoni A., Lo-Cascio, V. (1997) Effects of troglitazone on in vitro oxidation of LDL and HDL induced by copper ions and endothelial cells. Diabetologia 40:165-172.

Garg R., Kumbkami Y., Aljada A., Mohanty P., Ghanim H., Hamouda W., Dandona P. (2000) Troglitazone reduces reactive oxygen species generation by leukocytes and lipid peroxidation and improves flow-mediated vasodilatation in obese subjects. Hypertension 36(3):430-435.

Koya D., King J. (1998) Protein kinase C activation and the development of diabetic complications. Diabetes 47:859-866.

Kellerer M., Kroder G., Tippmer S., Berti L., Kiehn R., Mosthaf L., Haring H. (1994) Troglitazone prevents glucose-induced insulin resistance of insulin receptor in rat-1 fibroblasts. Diabetes 43:447-453.

Kroder G., Bossenmaier B., Kellerer M., Capp E., Stoyanov B., Muhihoefer L., Berti H., Horikoshi A., Ullrich A., Haring H. (1996) Tumor necrosis factor-alpha- and hyperglycemia-induced insulin resistance. Evidence for different mechanisms and different effects on insulin signaling. J. Clin. Invest. 27:97-108.

Bahr M., Spelleken M., Bock M., Von-Holtey R., Kiehn R., Eckel J. (1996) Acute and chronic effects of troglitazone (CS-045) on isolated rat ventricular cardiomyocytes. Diabetologia 39:766-774.

Kunisaki M., Bursell, S. E., Umeda F., Nawata H., King J. (1994) Normalization of diacylglycerol-protein kinase C activation by vitamin E in aorta of diabetic rats and cultured rat smooth muscle cells exposed to elevated glucose levels. Diabetes 43:1372-1377.

Tasinato A., Boscoboinik D., Bartoli G. M., Maroni P., Azzi A. (1995) d-α Tocopherol inhibition of vascular smooth muscle cell proliferation occurs at physiological concentrations, correlates with protein kinase C inhibition, and is independent of Its antioxidant properties. Proc. Natl. Acad. Sci. 92:12190-12194.

Higuchi Y., Matsukawa S. Glutathione depletion induces giant DNA and high-molecular-weight DNA fragmentation associated with apoptosis through lipid peroxidation and protein kinase C activation in C6 glioma cells. (1999) Arch Biochem Biophys. 363(1):33-42.

Watson D G, and Simpkins J W. (2001) Estradiol exposure inhibits PKC□ activation and ERK1/2 phosphorylation: potential mechanisms for its neuroprotective effects. Society for Neuroscience Abstracts, 27:655.9.

Kamencic H., Lyon A., Paterson P. G., and Juurlink H. J. (2000) Monochlorobimane fluorometric method to measure tissue glutathione. Analytical Biochemistry 286:35-37.

Davis J. S., Balinsky J. B., Harington J. S., Shepherd J. B. (1973) Assay, purification, properties and mechanism of action of γ-glutamylcysteine synthetase from the liver of rat and Xenopus laevis. Biochem. J. 133:667-678.

Oppenheimer L., Wellner V. P., Griffith O. W., Meister A. (1979) Glutathione synthetase purification from rat kidney and mapping of the substrate binding sites. J. Biol. Chem. 254:5184-5190.

Huang C. S., Anderson M. E., Meister A. (1993) Amino acid sequence and function of the light subunit of kidney gamma-glutamylcysteine synthetase. J. Biol. Chem. 268(27): 20578-20583.

Suzuki M., Mori M., Niwa., Hirata R., Ishikawa T. and Noyori R. (1997) Chemical implication for antitumor antiviral prostaglandins: reaction of Δ7-prostaglandin A1 and prostaglandin A1 methyl esters with thiol. J. Am. Chem. Soc. 119:2376-2385.

Green P. S., Gordon, K. and Simpkins J. W. (1997) Phenolic A ring requirement for the neuroprotective effects of steroids. J. Steroid Biochem. Molec. Biol. 63(4-6):229-235.

Satoh T., Furuta K., Tomokiyo K., Namura S., Nakatsuka D., Sugie Y., Ishikawa Y., Hatanaka H., Suzuki M., Watanabe Y. (2001) Neurotrophic actions of novel compounds designed from cyclopentenone prostaglandin. J. Neurochem. 77(1):50-62.

Crawford R. S., Mudaliar S. R., Henry R. R., Chait A. (1999) Inhibition of LDL oxidation in vitro but not ex vivo by troglitazone. Diabetes 48:783-790.

Markesbery W. R. (1997) Oxidative stress hypothesis in Alzheimer's disease. Free Radical Biology and Medicine 23(1):134-147.

What is claimed is:

1. A method for protecting a neuronal cell of a subject from a toxic insult, the method comprising: delivering an effective amount of a neuroprotective compound to the neuronal cell, wherein the neuroprotective compound comprises a phenolic ring moiety having a peroxisome proliferator-activated receptor-gamma (“PPAR-γ”) binding moiety associated therewith.

2. The method of claim 1 wherein the neuroprotective compound has a general structural formula of: 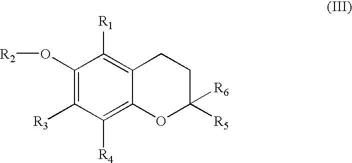
wherein,
R₁ is a hydrogen or a methyl group;
R₂ is hydrogen;
R₃ is hydrogen, methyl group, or tertiary butyl group;
R₄ is hydrogen or methyl group;
R₅ is hydrogen or methyl group; and
R₆ is hydrogen, an alkoxy-benzyl group, a an alkoxy benzyl thiazolidinedion group, or a 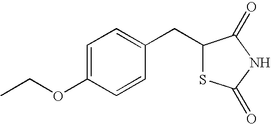
group.

3. The method of claim 1 wherein the neuroprotective compound has a general structural formula of: 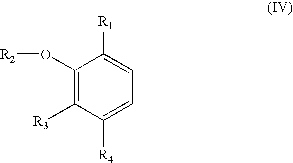
wherein,
R₁ is a hydrogen or a methyl group;
R₂ is hydrogen;
R₃ is hydrogen, methyl group, or tertiary butyl group;
R₄ is hydrogen or methyl group;

4. The method of claim 1, wherein the toxic insult comprises an acute neurodegenerative process.

5. The method of claim 4, wherein the acute neurodegenerative process characterized by toxic levels of hydrogen peroxide, or toxic levels of glutamate.

6. The method of claim 4, wherein the acute neurodegenerative process is a stroke, traumatic brain injury, schizophrenia, peripheral nerve damage, hypoglycemia, spinal cord injury, epilepsy, anoxia or hypoxia.

7. The method of claim 1, wherein the toxic insult comprises a chronic neurodegenerative disease.

8. The method of claim 7, wherein the chronic neurodegenerative disease is Alzheimer's disease, Parkinson's disease, Huntington's chorea, Pick's disease, diabetic peripheral neuropathy, multiple sclerosis, amyotrophic lateral sclerosis or aging.

9. The method of claim 1, wherein neuroprotective compound comprises:

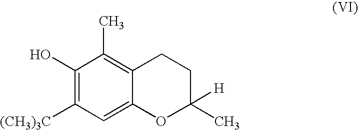

10. The method of claim 1, wherein neuroprotective compound comprises:

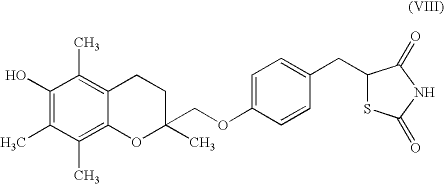

11. The method of claim 1, wherein neuroprotective compound comprises:

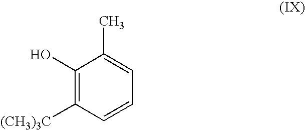

12. The method of claim 1, wherein the effective amount is in a range of 1 μM to 20 μM.

13. The method of claim 1, whereby delivering comprises parenterally delivering, whereby parenterally delivering comprises subcutaneous-(“SC”) delivering, intravascular-delivering or intramuscular-delivering.

14. The method of claim 1, wherein the neuroprotective compound is a PPAR-ligand selected from a group consisting of: 4-chloro-6-(2,3-xylidino)-2-pyrimidin-ylthio acetic acid; 5-[4-[(6-hydroxy-2,5,7,8-tetramethylchroman-2-yl)methoxy]-benzyl]-2,4-thiazolidinedione.

15. The method of claim 1, further consisting of a pharmaceutically acceptable salt, hydrate, ester, solvate, stereoisomer, or mixtures of stereoisomers of the neuroprotective compound.

16. A method for protecting a neuronal cell of a subject from a toxic insult, the method comprising: delivering an effective amount of a neuroprotective compound having a general structural formula of: 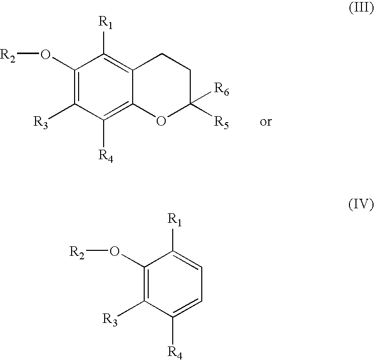
wherein,
R₁ is a hydrogen or a methyl group;
R₂ is hydrogen;
R₃ is hydrogen, methyl group, or tertiary butyl group;
R₄ is hydrogen or methyl group;
R₅ is hydrogen or methyl group; and
R₆ is hydrogen, an alkoxy-benzyl group, a an alkoxy benzyl thiazolidinedion group, or a 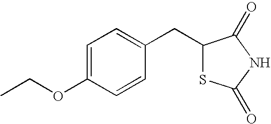
group.

17. The method of claim 16, wherein the toxic insult comprises an acute neurodegenerative process.

18. The method of claim 17, wherein the acute neurodegenerative process is toxic levels of hydrogen peroxide, or toxic levels of glutamate.

19. The method of claim 17, wherein the acute neurodegenerative process is a stroke, traumatic brain injury, schizophrenia, peripheral nerve damage, hypoglycemia, spinal cord injury, epilepsy, anoxia or hypoxia.

20. The method of claim 16, wherein the toxic insult comprises a chronic neurodegenerative disease.

21. The method of claim 20, wherein the chronic neurodegenerative disease is Alzheimer's disease, Parkinson's disease, Huntington's chorea, Pick's disease, diabetic peripheral neuropathy, multiple sclerosis, amyotrophic lateral sclerosis or aging.

22. The method of claim 16, wherein neuroprotective compound comprises:

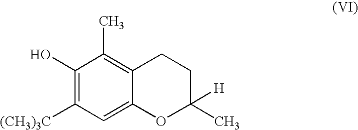

23. The method of claim 16, wherein neuroprotective compound comprises:

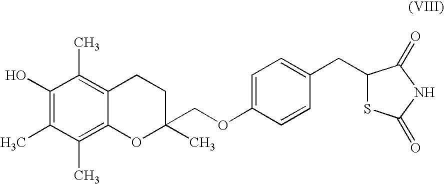

24. The method of claim 16, wherein neuroprotective compound comprises:

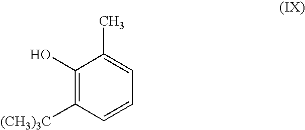

25. The method of claim 16, wherein the effective amount is in a range of 1 μM to 20 μM.

26. The method of claim 16, whereby delivering comprises parenterally delivering.

27. The method of claim 26, whereby parenterally delivering comprises subcutaneous-(“SC”) delivering, intravascular-delivering or intramuscular-delivering.

28. The method of claim 16, wherein the neuroprotective compound is a PPAR-ligand selected from a group consisting of: 4-chloro-6-(2,3-xylidino)-2-pyrimidin-ylthio acetic acid; 5-[4-[(6-hydroxy-2,5,7,8-tetramethylchroman-2-yl)methoxy]-benzyl]-2,4-thiazolidinedione.

29. A composition used for protecting a neuronal cell of a subject from a toxic insult, the composition comprising general structural formula of: 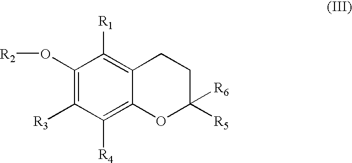
wherein,
R₁ is a hydrogen or a methyl group;
R₂ is hydrogen;
R₃ is hydrogen, methyl group, or tertiary butyl group;
R₄ is hydrogen or methyl group;
R₅ is hydrogen or methyl group; and
R₆ is hydrogen, an alkoxy-benzyl group, a an alkoxy benzyl thiazolidinedion group, or a 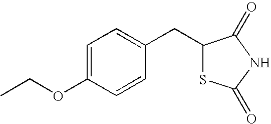
group.

30. The composition of claim 29, wherein the toxic insult comprises an acute neurodegenerative process.

31. The composition of claim 30, wherein the acute neurodegenerative process is toxic levels of hydrogen peroxide, or toxic levels of glutamate.

32. The composition of claim 30, wherein the acute neurodegenerative process is a stroke, traumatic brain injury, schizophrenia, peripheral nerve damage, hypoglycemia, spinal cord injury, epilepsy, anoxia or hypoxia.

33. The composition of claim 29, wherein the toxic insult comprises a chronic neurodegenerative disease.

34. The composition of claim 33, wherein the chronic neurodegenerative disease is Alzheimer's disease, Parkinson's disease, Huntington's chorea, Pick's disease, diabetic peripheral neuropathy, multiple sclerosis, amyotrophic lateral sclerosis or aging.

35. The composition of claim 29, wherein neuroprotective compound comprises:

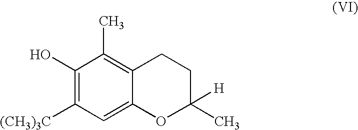

36. The composition of claim 29, wherein neuroprotective compound comprises:

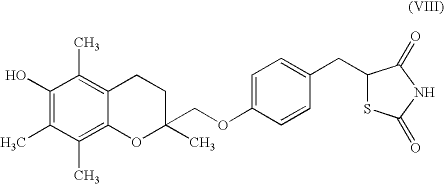

37. The composition of claim 29, wherein the effective amount is in a range of 1 μM to 20 μM.

38. A composition used for protecting a neuronal cell of a subject from a toxic insult, the composition comprising general structural formula of: 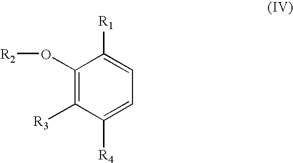
wherein,
R₁ is a hydrogen or a methyl group;
R₂ is hydrogen;
R₃ is hydrogen, methyl group, or tertiary butyl group;
R₄ is hydrogen or methyl group;

39. The composition of claim 38, wherein the toxic insult comprises an acute neurodegenerative process.

40. The composition of claim 39, wherein the acute neurodegenerative process is toxic levels of hydrogen peroxide, or toxic levels of glutamate.

41. The composition of claim 39, wherein the acute neurodegenerative process is a stroke, traumatic brain injury, schizophrenia, peripheral nerve damage, hypoglycemia, spinal cord injury, epilepsy, anoxia or hypoxia.

42. The composition of claim 38, wherein the toxic insult comprises a chronic neurodegenerative disease.

43. The composition of claim 42, wherein the chronic neurodegenerative disease is Alzheimer's disease, Parkinson's disease, Huntington's chorea, Pick's disease, diabetic peripheral neuropathy, multiple sclerosis, amyotrophic lateral sclerosis or aging.

44. The composition of claim 38, wherein neuroprotective compound comprises:

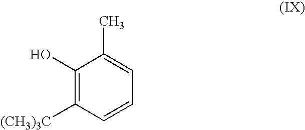

45. The composition of claim 38, wherein the effective amount is in a range of 1 μM to 20 μM.

46. The composition of claim 38, further consisting of a pharmaceutically acceptable salt, hydrate, ester, solvate, stercoisomer, or mixtures of stereoisomers of the neuroprotective compound.

47. A method for protecting a neuronal cell of a subject from a toxic insult, the method comprising: delivering an effective amount of a neuroprotective compound, wherein the neuroprotective compound comprises a prostaglandin (“PG”) having a peroxisome proliferator-activated receptor-gamma (“PPAR-γ”) binding moiety; and a cyclopentenone ring with a reactive α,β-unsaturated carbonyl group.

48. The method of claim 47, wherein the toxic insult comprises an acute neurodegenerative process.

49. The method of claim 48, wherein the acute neurodegenerative process is toxic levels of hydrogen peroxide, or toxic levels of glutamate.

50. The method of claim 48, wherein the acute neurodegenerative process is a stroke, traumatic brain injury, schizophrenia, peripheral nerve damage, hypoglycemia, spinal cord injury, epilepsy, anoxia or hypoxia.

51. The method of claim 47, wherein the toxic insult comprises a chronic neurodegenerative disease.

52. The method of claim 51, wherein the chronic neurodegenerative disease is Alzheimer's disease, Parkinson's disease, Huntington's chorea, Pick's disease, diabetic peripheral neuropathy, multiple sclerosis, amyotrophic lateral sclerosis or aging.

53. The method of claim 47, wherein the neuroprotective compound comprises the following structure:

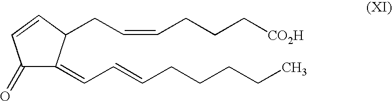

54. The method of claim 47, wherein the effective amount is in a range of 1 μM to 10 μM.

55. The method of claim 47, whereby delivering comprises parenterally delivering.

56. The method of claim 47, whereby parenterally delivering comprises subcutaneous-(“SC”) delivering, intravascular-delivering or intramuscular-delivering.

57. The method of claim 47, further consisting of a pharmaceutically acceptable salt, hydrate, ester, solvate, stereoisomer, or mixtures of stereoisomers of the neuroprotective compound.

58. A method for protecting a neuronal cell of a subject from a toxic insult, the method comprising: delivering an effective amount of a neuroprotective compound to the neuronal cell, wherein the neuroprotective compound comprises a phenolic ring moiety having a peroxisome proliferator-activated receptor-gamma (“PPAR-γ”) binding moiety associated therewith,
wherein
the neuroprotective compound has the structural formula: 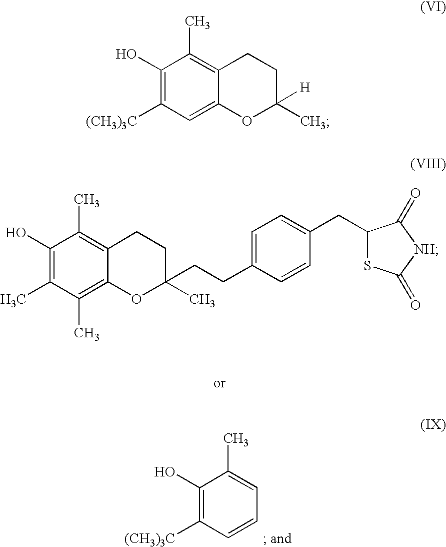
wherein,
the toxic insult comprises a neurodegenerative process having toxic levels of hydrogen peroxide, or toxic levels of glutamate.

59. The method of claim 58, wherein the neurodegenerative process is a stroke, traumatic brain injury, schizophrenia, peripheral nerve damage, hypoglycemia, spinal cord injury, epilepsy, anoxia, hypoxia, Alzheimer's disease, Parkinson's disease, Huntington's chorea, Pick's disease, diabetic peripheral neuropathy, multiple sclerosis, amyotrophic lateral sclerosis or aging.

60. A method for protecting a neuronal cell of a subject from a toxic insult, the method comprising: delivering an effective amount of a neuroprotective compound, wherein the neuroprotective compound comprises a prostaglandin (“PG”) having a peroxisome proliferator-activated receptor-gamma (“PPAR-γ”) binding moiety; and a cyclopentenone ring with a reactive α,β-unsaturated carbonyl group;
wherein the neuroprotective compound has the structural formula: 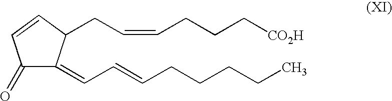
wherein,
the toxic insult comprises a neurodegenerative process having toxic levels of hydrogen peroxide, or toxic levels of glutamate.

61. The method of claim 60, wherein the neurodegenerative process is a stroke, traumatic brain injury, schizophrenia, peripheral nerve damage, hypoglycemia, spinal cord injury, epilepsy, anoxia, hypoxia, Alzheimer's disease, Parkinson's disease, Huntington's chorea, Pick's disease, diabetic peripheral neuropathy, multiple sclerosis, amyotrophic lateral sclerosis or aging.
